(19) United States
(12) Patent Application Publication
(10) Pub. No.: US 2020/0038454 A1
(43) Pub. Date: Feb. 6, 2020

(54) METHODS AND COMPOSITIONS FOR TISSUE THERAPY AND ANALYSIS

(71) Applicant: Ventrix, Inc., San Diego, CA (US)

(72) Inventors: Adam KINSEY, Grand Junction, CO (US); Paul CHAMBERLIN, La Jolla, CA (US)

(21) Appl. No.: 16/294,509

(22) Filed: Mar. 6, 2019

Related U.S. Application Data
(63) Continuation of application No. 16/032,459, filed on Jul. 11, 2018, now abandoned.
Application No. 16/032,459 is a continuation of application No. 14/771,194, filed on Aug. 27, 2015, now abandoned.
(60) Provisional application No. 61/770,885, filed on Feb. 28, 2013.

(51) Int. Cl. A61K 35/34; A61L 27/52; A61L 27/36; A61L 27/54; A61K 45/06

(52) U.S. Cl. CPC A61K 35/34; A61L 27/52; A61L 27/3633; A61L 27/54; A61L 2300/42; A61K 45/06; A61L 2430/20; A61L 2400/06; A61L 2300/422; A61L 27/367

(57) ABSTRACT

Methods and compositions comprising a biomaterial that are useful for treating a subject with a condition are described. More specifically, methods of treating a subject with a cardiovascular condition comprising delivering a biomaterial comprising extracellular matrix, and delivering a therapeutic cardiac device or therapeutic agents to the subject are described. In addition, methods of delivering extracellular matrix to a subject are also described.

### CROSS-REFERENCE TO RELATED APPLICATIONS

This application claims the benefit of U.S. Application Ser. No. 61/770,885, filed Feb. 28, 2013, which is hereby incorporated by reference in its entirety.

### BACKGROUND OF THE INVENTION

Cardiovascular disease (e.g., coronary heart disease) is the leading cause of death in the United States with approximately 400,000 annual incidences. Myocardial infarction (MI) is a common presentation of cardiovascular disease, and usually results from an acute interruption of blood supply, which is often caused by atherosclerotic plaque rupture with thrombus formation in a coronary vessel. Heart failure, which is the most common sequel of myocardial infarction, is the leading cause of hospital admission in the United States. Despite advances in medical, percutaneous, and surgical interventions, the 5-year survival rate of patients still remains around 50%.

MI results in an irreversible death of cardiomyocytes and extracellular matrix (ECM) degradation, followed by scar tissue formation. Eventually heart failure is onset, the heart dilates and remodels its tissue structure, leading to decreased functionality Cardiovascular tissue does not regenerate, therefore, current treatments for heart failure still rely heavily on invasive surgical procedures and do little to repair damaged heart tissue.

Cardiac regenerative medicine is emerging as a potential useful therapeutic strategy to restore cardiac function post MI. Cardiac progenitor cells or cardiac cells that have repair potential can be transplanted in vivo. However, without a proper biomaterial, its clinical implementation has been hampered by limited cell survival rate post transplantation and lack of cell engraftment in vivo. There is a need for a biomaterial that is biomimetic to heart extracellular matrix for cardiac repair to be delivered to a patient with cardiovascular disease, in conjunction with other cardiovascular therapeutic treatments.

### SUMMARY OF THE INVENTION

As noted above, there exists a pressing need for methods and compositions comprising a biomaterial that can be useful for treating a subject with a condition, such as cardiovascular condition. Similarly, there is a pressing need for delivering a biomaterial such as extracellular matrix to a subject. The present invention addresses these needs and provides related advantages as well.

In one aspect, the present invention provides a method of treating a subject with a cardiovascular condition. The method comprises injecting a biomaterial to the subject; and delivering to the subject one or more therapeutic agents that are configured to reduce a heart load, wherein the cardiovascular condition is improved following the injection of the biomaterial and the delivery of the one or more therapeutic agents.

In another aspect, the present invention provides another method of treating a subject with a cardiovascular condition. The method comprises injecting a biomaterial to the subject; and separately delivering a therapeutic cardiac device to the subject, wherein the cardiovascular condition is improved following the injection of the biomaterial and the delivery of the therapeutic cardiac device.

In some embodiments, the one or more therapeutic agents are selected from the group consisting of a blood pressure medication, an antiarrhythmic medication, a cholesterol-lowering drug, a blood thinner, an anticoagulant, a medication that controls heart rate, and a vasodilator.

In some embodiments, the biomaterial is injected into the left ventricle of the subject.

In some embodiments, the biomaterial comprises extracellular matrix (ECM) derived from a mammalian tissue. In some embodiments, the ECM is derived from a cardiac tissue. In some embodiments, the ECM is derived from a skeletal muscle tissue.

In some embodiments, the biomaterial comprises a hydrogel. In some embodiments, the therapeutic cardiac device comprises a hydrogel. In some embodiments, therapeutic cardiac device is a coronary stent. In some embodiments, the therapeutic cardiac device is a left ventricular assist device. In some embodiments, the therapeutic cardiac device is a pace maker.

In some embodiments, the injecting of the biomaterial is provided through a cardiac catheter with a femoral artery access.

In some embodiments, the improvement of the cardiovascular condition is greater than 50%. In some embodiments, the improvement of the cardiovascular condition is greater than the improvement from treating the subject with the biomaterial alone or with the one or more therapeutic agents alone. In some embodiments, the improvement of the cardiovascular condition is greater than the improvement from treating the subject with the biomaterial alone or the therapeutic cardiac device alone. In some embodiments, the biomaterial and the one or more therapeutic agents act upon the cardiovascular condition synergistically. In some embodiments, the biomaterial and the therapeutic cardiac device act upon the cardiovascular condition synergistically.

In some embodiments, the cardiovascular condition is a condition selected from the group consisting of: coronary heart disease, cardiomyopathy, myocardial infarction, hypertensive heart disease, heart failure, cor pulmonale, cardiac dysrhythmias, inflammatory heart disease, valvular heart disease, stroke and cerebrovascular disease, and peripheral arterial disease.

In some embodiments, the cardiovascular condition is coronary heart disease. In some embodiments, the cardiovascular condition is cardiomyopathy. In some embodiments, the cardiovascular condition is heart failure. In some embodiments, the cardiovascular condition is peripheral arterial disease. In some embodiments, the cardiovascular condition is myocardial infarction.

In some embodiments, the biomaterial and the one or more therapeutic agents are delivered separately. In some embodiments, the biomaterial and the one or more therapeutic agents are delivered together.

In some embodiments, the therapeutic cardiac device is designed to prevent flow constriction in a blood vessel. In some embodiments, the therapeutic cardiac device is designed to prevent ischemia. In some embodiments, the biomaterial further comprises an angiographic contrast agent.

In yet another aspect, the current invention provides a method of delivering extracellular matrix (ECM) to a subject. The method comprises delivering a first composition in a liquid form to the subject, wherein the first composition comprises the ECM or components derived from the ECM; and delivering a second composition in a liquid to the subject such that a portion of the second composition contacts a portion of the first composition within the subject; wherein the ECM forms a gel after the contacting of the first composition with the second composition.

In another aspect, provided herein is a method of treating a subject with an acute ST-elevation myocardial infarction (STEMI) comprising injecting a biomaterial to the heart of the subject, wherein the total volume of the biomaterial injected is 1-10 ml. In some embodiments, the biomaterial is injected into the infarct of the subject. In certain embodiments, the injecting of the biomaterial is provided through a cardiac catheter with a femoral artery access. In some embodiments, the subject has a left ventricular ejection fraction of 25%-55%. In certain embodiments, the biomaterial comprises extracellular matrix (ECM) derived from a cardiac tissue.

In certain embodiments of the method of treating a subject with an acute ST-elevation myocardial infarction (STEMI), the method further comprises conducting a diagnostic imaging procedure to the subject prior to the injection. In some embodiments, the diagnostic imaging procedure is conducted less than 2 weeks after the myocardial infarction.

In certain embodiments of the method of treating a subject with an acute ST-elevation myocardial infarction (STEMI), the subject is separately delivered a therapeutic cardiac device. In some embodiments, the therapeutic cardiac device is a percutaneous coronary intervention device. In certain embodiment, the subject shows improvement in cardiac function after the injection of the biomaterial, and wherein the improvement of the cardiovascular condition is greater than the improvement from treating the subject with the biomaterial alone or the therapeutic cardiac device alone. In some embodiments, the biomaterial and the therapeutic cardiac device act upon the cardiovascular condition synergistically.

In certain embodiments of the method of treating a subject with an acute ST-elevation myocardial infarction (STEMI), the subject is further administered one or more therapeutic agents that are configured to reduce a heart load. In some embodiments, the one or more therapeutic agents are selected from the group consisting of a blood pressure medication, an antiarrhythmic medication, a cholesterol-lowering drug, a blood thinner, an anticoagulant, a medication that controls heart rate, and a vasodilator. In certain embodiments, the biomaterial and the one or more therapeutic agents are delivered separately. In some embodiments, the subject shows improvement in cardiac function after the injection of the biomaterial, and wherein the improvement is greater than the improvement from treating the subject with the biomaterial alone or with the one or more therapeutic agents alone. In certain embodiments, the biomaterial and the one or more therapeutic agents act upon the cardiovascular condition synergistically.

In some embodiments of the method of treating a subject with an acute ST-elevation myocardial infarction (STEMI), the subject shows improvement in ejection fraction after the injection of the biomaterial. In certain embodiments, the biomaterial increases cell influx in the infarct. In some embodiments, the left ventricular geometry of the subject is preserved. In certain embodiments, after the injection of the biomaterial, the subject does not enter heart failure within 5 years.

In a further aspect, provided herein is a method of delivering extracellular matrix (ECM) to a subject comprising: (a) delivering a first composition in a liquid form to the subject, wherein the first composition comprises the ECM or components derived from the ECM; and (b) delivering a second composition in a liquid to the subject such that a portion of the second composition contacts a portion of the first composition within the subject, wherein the ECM forms a gel after the contacting of the first composition with the second composition.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary method of treating a cardiovascular condition, wherein Standard Therapy is treatment with a cardiac therapeutic device or a therapeutic agent, and Combined Therapy is Standard Therapy combined with a biomaterial injection.

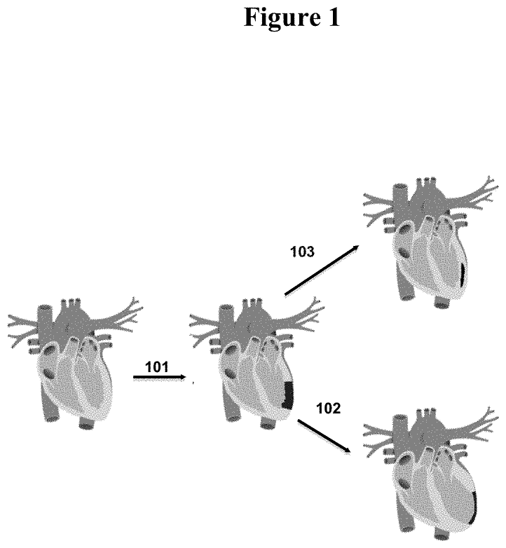

### DETAILED DESCRIPTION OF THE INVENTION

Currently, common treatments known in the art are heart transplantation, left ventricular (LV) assist devices, stents, and/or current pharmaceutical regimens. After myocardial infarction, current standard therapies such as pharmaceuticals and therapeutic devices (or lack of therapy) are generally not effective enough and eventually lead to death to the cardiomyocytes, negative LV remodeling, LV dilation, and heart failure. Furthermore, current standard therapies are not adequate to prevent negative LV remodeling and may introduce further iatrogenic damages. Therefore, development of new therapies that can further improve and repair heart functions upon current standard therapies for end-stage heart failure is needed.

A method of treating a subject with a cardiovascular condition by delivering an injectable biomaterial composition is described herein. In some instances, delivering a biomaterial herein to a LV can provide increased regeneration, reduced infarct size, reduced LV remodeling, or improved cardiac function. The solution form, gel form, and adsorbed form of the heart matrix provide many of the constituents of native ECM at similar ratios found in vivo.

In some instances, the biomaterial comprising heart ECM can be used for cell therapy. Cell therapy, the injection of healthy cells into the left ventricle (LV) infarct wall in an attempt to regenerate and repair the damaged myocardium, has been investigated recently. However, most pre-clinical studies have shown poor cell engraftment and survival post-transplantation without delivery along with a proper matrix. Native cardiac cells exist in a highly complex extracellular milieu in vivo; and an ECM that more closely mimics this native environment may be beneficial for cultured cell survival and function/maturation in vivo.

More recently, acellular hydrogel biomaterials have shown great promise in providing similar functional benefit without the complications associated with cell delivery. Injection of a gel-like biomaterial can provide structure support for the increase in heart loads and prevent negative LV remodeling post MI. Biomaterial products for cardiac therapy have been limited because few have been manufactured specifically for the myocardium. Materials currently under investigation for injection into the myocardium include, without limitation, fibrin, collagen, alginate, matrigel, and gelatin.

Some naturally derived materials are currently being investigated for injection into the myocardium including fibrin, collagen, alginate, matrigel, and gelatin. None of these provide a significant amount of the native components of the heart extracellular matrix. For arrhythmia treatment, current non-ablative forms include injection of alginate, fibrin and cells. Existing matrices for in vitro cell culture for cardiomyocytes, stem cells, and other cardiac relevant cells include collagen, laminin, SureCoat (Cellutron, mixture of collagen and laminin), Matrigel, and gelatin.

Methods and/or compositions of the invention comprising a biomaterial, such as a hydrogel and/or decellularized cardiac extracellular matrix (ECM), and/or the use thereof described herein may be useful for purposes described herein, such as treating, supporting, maintaining, enhancing, ameliorating, and/or improving health, cardiac function, cardiovascular function, cardiac tissue regeneration and/or cardiac repair of a subject with a condition (e.g., cardiovascular condition). The cardiovascular condition can be myocardial infarction, or a STEMI. The subject can be concurrently or separately administered with other therapeutic treatments or therapeutics. The biomaterial can be decellularized cardiac extracellular matrix (ECM) or decellularized skeletal muscle ECM. A description of various aspects, features, embodiments, and examples, is provided herein.

It will be understood that a word appearing herein in the singular encompasses its plural counterpart, and a word appearing herein in the plural encompasses its singular counterpart, unless implicitly or explicitly understood or stated otherwise. Further, it will be understood that for any given component described herein, any of the possible candidates or alternatives listed for that component, may generally be used individually or in any combination with one another, unless implicitly or explicitly understood or stated otherwise. Additionally, it will be understood that any list of such candidates or alternatives, is merely illustrative, not limiting, unless implicitly or explicitly understood or stated otherwise. Still further, it will be understood that any figure or number or amount presented herein is approximate, and that any numerical range includes the minimum number and the maximum number defining the range, whether the word “inclusive” or the like is employed or not, unless implicitly or explicitly understood or stated otherwise. Generally, the term “approximately” or “about” or the symbol “.about.” in reference to a figure or number or amount includes numbers that fall within a range of .+−0.5% of same, unless implicitly or explicitly understood or stated otherwise. Yet further, it will be understood that any heading employed is by way of convenience, not by way of limitation. Additionally, it will be understood that any permissive, open, or open-ended language encompasses any relatively permissive to restrictive language, less open to closed language, or less open-ended to closed-ended language, respectively, unless implicitly or explicitly understood or stated otherwise. Merely by way of example, the word “comprising” may encompass “comprising”-, “consisting essentially of”-, and/or “consisting of”-type language.

Generally, the term “concurrent administration” is in reference to two or more subjects of administration for administration to a subject body, such as components, agents, substances, materials, compositions, devices, systems and/or the like, refers to administration performed using dose(s) and time interval(s) such that the subjects of administration are present together within the subject body, or at a site of action in the subject body, over a time interval in less than de minimus quantities. The time interval may be any suitable time interval, such as an appropriate interval of minutes, hours, days, or weeks, for example. The subjects of administration may be administered together, such as parts of a single composition, for example, or otherwise. The subjects of administration may be administered substantially simultaneously (such as within less than or equal to about 5 minutes, about 3 minutes, or about 1 minute, of one another, for example) or within a short time of one another (such as within less than or equal to about 1 hour, 30 minutes, or 10 minutes, or within more than about 5 minutes up to about 1 hour, of one another, for example). The subjects of administration so administered may be considered to have been administered at substantially the same time. One of ordinary skill in the art will be able to determine appropriate dose(s) and time interval(s) for administration of subjects of administration to a subject body so that same will be present at more than de minimus levels within the subject body and/or at effective concentrations within the subject body. When the subjects of administration are concurrently administered to a subject body, any such subject of administration may be in an effective amount that is less than an effective amount that might be used were it administered alone. The term “effective amount,” which is further described herein, encompasses both this lesser effective amount and the usual effective amount, and indeed, any amount that is effective to elicit a particular condition, effect, and/or response. As such, a dose of any such subject of concurrent administration may be less than that which might be used were it administered alone. One or more effect(s) of any such subject(s) of administration may be additive or synergistic. Any such subject(s) of administration may be administered more than one time.

Generally, the terms “deliver”, “administrate”, “give”, or “apply” are in reference to the act of administration of a subject that is separate from a subject body to inside of a subject body, or in direct contact with the subject body; or are in reference to administration of an act of a procedure directly or indirectly onto the subject body. The state of the subject of administration may change according to the environment wherein the subject of administration is delivered into. The term “environment,” which is further described herein, encompasses one or more parameters that measure different aspects of the environment, such as temperature, pressure, humidity, osmolarity, concentration, shear stress, compression, tension, collision, hematocrit, dimensions of space, types of subject that may be adjacent to, types of agents that may be in contact with, and the like. The subject of administration can be delivered through different routes of delivery. The subject of administration can be delivered through injection. The state or the environment of the subject body may change or may not change upon administration of the subject of administration or the act of a procedure.

The biomaterial (e.g., ECM) can be administered, delivered, applied, and/or injected to a subject separately or concurrently with one or more therapeutics that are known in the medical art. To treat a subject with a condition, the types of therapeutic treatment that can be administered, delivered, applied, and/or given to a subject concurrently with a biomaterial (e.g., ECM) can be therapeutic agents, medical devices or therapeutic cardiac devices, biomaterials, cells, reporter agents, and/or surgical procedures. The subject with a condition can be treated with one or more types of therapeutics at the same time or sequentially. The therapeutics can be delivered to the subject with a condition together in one procedure, or in one composition, with the biomaterial. The therapeutics can be delivered separately with the biomaterial. The biomaterial described herein can further comprise an additional component, for example without limitation: a cell, a peptide, polypeptide, or protein, a nucleic acid such as a polynucleotide or oligonucleotide, DNA, RNA, a vector expressing a DNA of a bioactive molecule, polymer or other material, survival-promoting additives, crosslinkers, proteoglycans, glycosaminolycans, and other additives like nutrients or therapeutic agents (e.g., drug) molecules. One additional component can be included in the biomaterial or several. In addition, where proteins such as growth factors are added into the extracellular matrix biomaterial, the proteins may be added into the biomaterial, or the protein molecules may be covalently or non-covalently linked to a molecule in the biomaterial. The covalent linking of protein to matrix molecules can be accomplished by standard covalent protein linking procedures known in the art. The protein may be covalently linked to one or more matrix molecules. In some instances, the biomaterial also comprises an immunosuppressive agent. In other cases, the composition does not comprise an immunosuppressive agent.

Methods of treating a subject with a condition (e.g., cardiovascular condition) described in the invention may include the steps consisting of: injecting a biomaterial to the subject. The methods can further comprise delivering to the subject one or more therapeutic agents. The cardiovascular condition can be improved following the methods. In some cases, the therapeutic agent is delivered as part of the extracellular matrix composition, as a component of an injected solution of ECM, or in a separate composition; in some cases the therapeutic agent is delivered concurrently, before, or after the delivery of a biomaterial described herein.

The one or more therapeutic agents or drugs may be configured to treat the cardiovascular condition, such as to reduce a heart load. The types of therapeutic agents can be cardiac drugs, immunosuppressive agents, pain killers, antibiotics, pro-survival small molecules, growth factors, or plate-rich plasma. The therapeutic agents can be selected from therapeutics agents known in the medical art as described herein. Examples of cardiac drugs, or therapeutic agents that are configured to reduce the heart load, include, without limitation: blood pressure or hypertension medications (e.g., ACE inhibitors, alpha agonists, alpha blockers, Angiotensin II receptor blockers, diuretics, or renin blockers); antiarrhythmics (e.g., sodium channel blockers, beta blockers, potassium channel blockers, calcium channel blockers); cholesterol lowering drugs (e.g., statins, chloestyramine); blood thinners or anticoagulants (e.g., aspirin, aprotinin, clopidogrel, enoxaparin, heparin, warfarin, dabiagatran etexilate); medications that control heart rate (e.g., digitalis preparations); vasodilators (e.g., nitroglycerin) and/or thrombolytic agents. Non-limiting examples of diuretics include: Acetazolamide-Diamox, Chlorthalidone-Thalitone, Hydrochlorothiazide-HydroDiuril, also sold as Microzide and Esidrix, Indapamide-Lozol, Metolazone-Zaroxolyn, also sold as Mykrox, Amiloride hydrochloride-Midamor, Bumetanide-Bumex, Ethacrynic acid-Edecrin, Furosemide-Lasix, Spironolactone-Aldactone, Torsemide-Demadex, and Triamterene-Dyrenium. Non-limiting examples of beta blockers include: Acebutolol-Sectral, Atenolol-Tenormin, Betaxolol-Kerlone, Bisoprolol-Zebeta, also sold as Ziac, Carteolol-Cartrol, Carvedilol-Coreg, Labetalol-Normodyne, also sold as Trandate, Metoprolol-Lopressor, also sold as Toprol, Nadolol-Corgard, Penbutolol-Levatol, Propranolol-Inderal, Inderal LA, and Timolol-Blocadren. Non-limiting examples of calcium channel blockers include: Amlodipine-Norvasc, also sold as Caduet and Lotrel, Diltiazem-Cardizem, also sold as Dilacor and Tiazac, Felodipine-Plendil, Isradipine-DynaCirc, Nicardipine-Cardene, Nifedipine-Procardia XL, also sold as Adalat, Nisoldipine-Sular, Verapamil hydrochloride-Isoptin, also sold as Calan, Verelan, and Covera. Non-limiting examples of ACE inhibitors include: Benazepril-Lotensin, Captopril-Capoten, Enalapril-Vasotec, also sold as Vaseretic, Fosinopril-Monopril, Lisinopril-Prinivil, also sold as Zestril, Moexipril-Univasc, Quinapril-Accupril, Ramipril-Altace, and Trandolapril-Mavik. Non-limiting examples of angiotensin II receptor blockers include: Candesartan-Atacand, Irbesartan-Avapro, Losartan-Cozaar, Telmisartan-Micardis, and Valsartan-Diovan. Non-limiting examples of blood pressure or hypertension medications can also include: Clonidine-Catapres, Doxazosin-Cardura, Guanabenz-Wytensin, Guanfacine-Tenex, Hydralazine hydrochloride-Apresoline, Methyldopa-Aldomet, Prazosin-Minipress, Reserpine-Serpasil, and Terazosin-Hytrin. Blood pressure or hypertension medications can also be combination drugs, such as: Amiloride and hydrochlorothiazide-Moduretic, Amlodipine and benazepril-Lotrel, Atenolol and chlorthalidone-Tenoretic, Benazepril and hydrochlorothiazide-Lotensin HCT, Bisoprolol and hydrochlorothiazide-Ziac, Captopril and hydrochlorothiazide-Capozide, Enalapril and hydrochlorothiazide-Vaseretic, Felodipine and enalapril-Lexxel, Hydralazine and hydrochlorothiazide-Apresazide, Lisinopril and hydrochlorothiazide-Prinzide, also sold as Zestoretic, Losartan and hydrochlorothiazide-Hyzaar, Methyldopa and hydrochlorothiazide-Aldoril, Metoprolol and hydrochlorothiazide-Lopressor HCT, Nadolol and bendroflumethiazide-Corzide, Propranolol and hydrochlorothiazide-Inderide, Spironolactone and hydrochlorothiazide-Aldactazide, Triamterene and hydrochlorothiazide-Dyazide, also sold as Maxide, and Verapamil extended release) and trandolapril-Tarka. Non-limiting examples of cholesterol lowering drugs include: atorvastatin, fluvastatin, pravastatin, lovastatin, simvastatin, rovuvastatin, lovastatin, lovastatin/niacin ER, nicotinic acid, niacin XR, fibrates, fenofibrate and micronized fenofibrate. Non-limiting examples of antiarrhythmics include: Betapace/Betapace AF (sotalol), Blocadren (timolol), Calan/Calan SR (verapamil), Cardioquin (quinidine), Cardizem (diltiazem), Cartia (diltiazem), Cordarone (amiodarone), Coreg/Coreg CR (carvedilol), Corgard (nadolol), Covera(verapamil), Dilacor XR (diltiazem), Diltia XT (diltiazem), Inderal/Inderal LA (propranolol), Inderide (propranolol), Innopran XL (propranolol), Isoptin (verapamil), Kerlone (betaxolol), Lopressor/Lopressor HCT (metoprolol), Mexitil (mexiletine), Norpace/Norpace CR (disopyramide), Pacerone (amiodarone), Procanbid (procainamide), Pronestyl (procainamide), Quinaglute Dura-tabs (quinidine), Quinidex Extentabs (quinidine), Quinora (quinidine), Rythmol (propafenone), Sectral (acebutolol), Sorine (sotalol), Tambocor (flecainide), Tenormin (atenolol), Tiazac (diltiazem), Tikosyn (dofetilide), Timolide (timolol), Toprol XL (metoprolol), Verelan/Verelan PM (verapamil), and Zebeta (bisoprolol).

In some instances, the type of therapeutic agents that can be delivered to a subject separately or concurrently with a biomaterial (e.g., ECM) can be immunosuppressive agents, or immunosuppressants. Examples of categories of immunosuppressive agents include, without limitation: glucocorticoids, cytostatics (e.g., alkylating agents, antimetabolites, methotrexate, azathioprine and mercaptopurine, or cytotoxic antibiotics), antibodies (e.g., polyclonal antibodies, monoclonal antibodies, and/or IL-2 receptor directed antibodies), drugs acting on immunophilins (e.g., ciclosporin, tacrolimus, or sirolimus), interferons, opioids, TNF binding proteins, mycophenolate, and/or small biological agents. Non-limiting examples of immunosuppressive agents can be selected from the group consisting of: Abatacept, Abetimus, Adalimumab, Afelimomab, Alefacept, Anakinra, Anti-IL-6, Anti-thymocyte globulin, Ascomycin, Azathioprine, Basiliximab, Belimumab, Briakinumab, CDP323, Certolizumab pegol, Ciclosporin, Cyclosporins, Daclizumab, 4-Deoxypyridoxine, Discovery and development of thalidomide and its analogs, Disodium aurothiomalate, Eculizumab, Efalizumab, Eritoran, Etanercept, Everolimus, Fingolimod, Gliotoxin, Gusperimus, Template:Immunosuppressants, Infliximab, Laquinimod, Leflunomide, Lenalidomide, Mapracorat, Mepolizumab, Methotrexate, Mizoribine, Muromonab-CD3, Mycophenolate mofetil, Mycophenolic acid, Natalizumab, Neurovax, Pegsunerceptm, Pimecrolimus, Pomalidomide, ReciGen, Ridaforolimus, Rilonacept, Secukinumab, Selective glucocorticoid receptor agonist, Sirolimus, Tacrolimus, Teriflunomide, Thalidomide, Tocilizumab, Umirolimus, Ustekinumab, Voclosporin, and Zotarolimus.

In some instances, the type of therapeutic agents that can be delivered to a subject separately or concurrently with a biomaterial (e.g., ECM) can be anti-inflammatory drugs. Examples of anti-inflammatory drugs include, but not limited to: steroids, non-steroidal anti-inflammatory drugs, Immune Selective Anti-Inflammatory Derivatives (ImSAIDs), or Herbs.

In some instances, the type of therapeutic agents that can be delivered to a subject separately or concurrently with a biomaterial (e.g., ECM) can be pain-killers. Examples of pain-killers include, but are not limited to: aspirin, acetaminophen, ibuprofen, naproxen sodium, ketoprofen, celecoxib, tramadol, meperidine HCl, hydrocodone, hydrocodone-APAP, oxycodone HCl, terephthalate, morphine sulfate, fentanyl, hydromorphone hydrochloride, dihydromorphinone, and oxymorphone.

In some instances, the type of therapeutic agents that can be delivered to a subject separately or concurrently with a biomaterial (e.g., ECM) can be antibiotics. Non-limiting examples of antibiotics include: penicillins (e.g., penicillin, amoxicillin), cephalosporins (e.g., cephalexin), macrolides (e.g., erythromycin, clarithromycin, azithromycin), fluoroquinolones (e.g., ciprofloxacin, levofloxacin, ofloxacin), sulfonamides (e.g., co-trimoxazole, trimethoprim), tetracyclines (e.g., tetracycline, doxycycline), and aminoglycosides (e.g., gentamicin, tobramycin).

In some instances, the type of therapeutic agents that can be delivered to a subject separately or concurrently with a biomaterial (e.g., ECM) can be pro-survival small molecules. Non-limiting examples of pro-survival small molecules include: ROCK inhibitor Y-27632, B-Cell Lymphoma 2 Proteins, NSC23766, and DDD00033325.

In some instances, the type of therapeutic agents that can be delivered to a subject separately or concurrently with a biomaterial (e.g., ECM) can be growth factors. Non-limiting examples of growth factors include: erythropoietin (EPO), angiopoietin (Ang), stem cell factor (SCF), vascular endothelial growth factor (VEGF), fibroblast growth factor (FGF), nerve growth factor (NGF), hematopoietic cell growth factor, hepatocyte growth factor, hepatoma-derived growth factor, migration-stimulating factor, autocrine motility factor, epidermal growth factor (EGF), insulin-like growth factor 1 (IGF-1), transforming growth factor (TGF), cartilage growth factor (CGF), keratinocyte growth factor (KGF), skeletal growth factor (SGF), osteoblast-derived growth factor (BDGF), cytoline growth factor (CGF), colony stimulating factor (CSF), integrin modulating factor (IMF), platelet-derived growth factor (PDGF), calmodulin, bone morphogenic proteins (BMP), and tissue inhibitor matrix metalloproteinase (TIMP). In some instances, the type of therapeutic agents that can be delivered to a subject concurrently with a biomaterial (e.g., ECM) can be platelet rich plasma (PRP).

In some instances, a biomaterial (e.g., ECM) may be injected to a subject separately or concurrently with microbeads. Microbeads can be a part of the biomaterial or delivered by the biomaterial. Exemplary microbeads can be any variety of materials, for example, natural or synthetic. In some instances, the microbeads can have varied degradation properties or comprise, for example, MMP inhibitors, growth factors, or small molecules.

Methods of treating a subject with a condition (e.g., cardiovascular condition) described in the invention may include the steps consisting of: injecting a biomaterial (e.g., ECM) to the subject, and further comprising delivering to the subject one or more therapeutics. The one or more therapeutics can be any therapeutic known in the medical art that can be used to treat cardiovascular conditions. The types of therapeutics that can be delivered to a subject with a condition (e.g., cardiovascular condition) can be a medical device or therapeutic cardiac device. The cardiovascular condition can be improved following the injection of the biomaterial and the delivery of the one or more therapeutics. In some cases, the therapeutic agent is delivered as part of the extracellular matrix composition, as a component of an injected solution of ECM, or in a separate composition; in some cases the therapeutic agent is delivered concurrently, before, or after the delivery of a biomaterial described herein.

The therapeutic cardiac device can be delivered to a subject with a cardiovascular condition separately with a biomaterial that is injected to the subject, such that the cardiovascular condition is improved following the injection of the biomaterial and the delivery of the therapeutic cardiac device. In some embodiments, the therapeutic cardiac device may be designed to prevent flow constriction in a blood vessel. In some embodiments, the therapeutic cardiac device may be designed to prevent or treat ischemia. Non-limiting examples of the therapeutic cardiac device include: implantable cardiac defibrillator (ICD), internal monitoring devices, pacemakers (or pacemaker leads), the left ventricular assist device (LVAD), heart valves, endovascular grafts, pacing devices, endovascular stents (e.g., coronary stent), devices for rhythm management, devices for septal defects and management, devices for coronary heart disease management, devices for congestive heart failure, and cardiopulmonary bypass system. In some embodiments, the therapeutic cardiac device may comprise a biomaterial (e.g., a hydrogel).

As described herein, a subject with a condition (e.g., cardiovascular condition) can be treated by injecting a biomaterial (e.g., ECM) to the subject. The biomaterial can be delivered to a subject with a condition (e.g., cardiovascular condition) separately or concurrently with another type of biomaterial. The biomaterial can comprise one or more types of biomaterials. Types of biomaterials include synthetic or naturally occurring polymers, hydrogel, metal, ceramic, ECM, or tissue grafts. In some embodiments, the biomaterial may comprise a hydrogel.

In some instances, the biomaterial can comprise synthetic or naturally occurring polymer. Exemplary polymers described herein include, but are not limited to: polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethelene, nitinol, cellulose or methylcellulose, and collagen from animal and non-animal sources (such as plants or synthetic collagens). In some instances, a polymer is biocompatible, biodegradable and/or bioabsorbable, bioactive, biointegrative, and/or bioconductive. Exemplary biodegradable or bioabsorbable polymers include, but are not limited to: polylactides, poly-glycolides, polycarprolactone, polydioxane and their random and block copolymers. A biodegradable and/or bioabsorbable polymer can contain a monomer selected from the group consisting of a glycolide, lactide, dioxanone, caprolactone, trimethylene carbonate, ethylene glycol and lysine. The material can be a random copolymer, block copolymer or blend of monomers, homopolymers, copolymers, and/or heteropolymers that contain these monomers. The biodegradable and/or bioabsorbable polymers can contain bioabsorbable and biodegradable linear aliphatic polyesters such as polyglycolide (PGA) and its random copolymer poly(glycolide-co-lactide-) (PGA-co-PLA). Other examples of suitable biocompatible polymers are polyhydroxyalkyl methacrylates including ethylmethacrylate, and hydrogels such as polyvinylpyrrolidone and polyacrylamides. Other suitable bioabsorbable materials are biopolymers which include collagen, gelatin, alginic acid, chitin, chitosan, fibrin, hyaluronic acid, dextran, polyamino acids, polylysine and copolymers of these materials. Any combination, copolymer, polymer or blend thereof of the above examples is contemplated for use according to the invention. Such bioabsorbable materials may be prepared by known methods.

In some instances, the biomaterial herein may comprise a naturally derived polymer. Examples of naturally derived polymers for use herein include, but are not limited to: alginate, fibrin glue, algarose, chitosan, gelatin, and polysaccharides such as hyaluronic acid and any combination thereof. In an instance, a biomaterial here comprises an alginate bead that is coated with an ECM biomaterial as described herein.

In an instance, the biomaterial herein can comprise a biocompatible metal. An example of biocompatible metal includes, but is not limited to, titanium. In an example, a biomaterial herein comprises small diameter fibers or small diameter particles of a biocompatible metal. The metal within the biomaterial can provide support to the material structure. In addition, when the decellularized ECM degrades in vivo, the metal portions of the composition can be left behind in order to provide a support structure for the surrounding tissue.

In some instances, the biomaterial herein can comprise cellulose or methylcellulose. Cellulose can be utilized to form the material into a desired shape both. In another aspect herein, a device is provided, wherein cellulose provides a substrate on which a biomaterial as described herein is deposited. The device can then be delivered in a particular shape for tissue repair.

Biomaterials comprising native extracellular matrix scaffolds have been prepared for use in mammals in tissue grafts procedures. Examples of the ECM matrix include without limitation: small intestine submucosa (SIS) such as the scaffolds described in U.S. Pat. No. 5,275,826, urinary bladder submucosa (UBS) such as the scaffolds described in U.S. Pat. No. 5,554,389, stomach submucosa (SS) such as the scaffolds described in U.S. Pat. No. 6,099,567, and liver submucosa (LS) or liver basement membrane (LBM) such as the scaffolds described in U.S. Pat. No. 6,379,710. In addition, collagen from mammalian sources can be retrieved from matrix containing tissues and used to form a matrix composition. Extracellular matrices can also be synthesized from cell cultures. Heart decellularization has been published for the purpose of regrowing an entire heart (Ott et al, Nature Medicine, 2008). An injectable gel form of porcine bladder matrix has also been described (Freytes et al, Biomaterials, 2008. Commercially available ECM preparations can also be combined in the methods described herein. In one embodiment, the biomaterial can comprise biomaterial that is derived from small intestinal submucosa or SIS. Commercially available preparations include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). In another embodiment, the biomaterial can be ECM that is derived from dermis. Commercially available preparations include, but are not limited to Pelvicol™ (sold as Permacol™ in Europe; Bard, Covington, Ga.), Repliform™ (Microvasive; Boston, Mass.) and Alloderm™ (LifeCell; Branchburg, N.J.).

As described herein, a subject with a condition (e.g., cardiovascular condition) can be treated by injecting a biomaterial (e.g., ECM) to the subject separately or concurrently with the delivery of one or more therapeutics. The therapeutics can be therapeutic treatments. The therapeutic treatment that can be delivered to, or applied on a subject with a condition (e.g., cardiovascular condition) can be a surgical or medical procedure. The surgical or medical procedure can be applied concurrently or separately with the injection of the biomaterial. Non-limiting examples of medical procedures that can be applied on a subject with a cardiovascular condition include cardiac resynchronization therapy, laser angioplasty, artificial heart valve surgery, atherectomy, bypass surgery or CABG or open heart surgery, cardiomyoplasty, heart transplant, minimally invasive heart surgery (or limited access coronary artery surgery, radiofrequency ablation (or catheter ablation), stent procedure, transmyocardial revascularization (TMR), percutaneous coronary interventions (PCI) devices, and balloon angioplasty.

In an embodiment, a therapeutic treatment that can be delivered to a subject with a condition (e.g., cardiovascular condition) can be cell therapy. In a cell therapy, a plurality of one or more types of cell can be delivered in situ or in vivo. The cells can be delivered in a composition that also comprises the biomaterial, or separately with the biomaterial. The cells can be from cell sources for treating the myocardium that include autologous, non-autologous, HLA-matched, allogeneic, xenogeneic, or autogeneic sources. Accordingly, human embryonic stem cells (hESC), neonatal cardiomyocytes, myofibroblasts, mesenchymal cells, autotransplanted expanded cardiomyocytes, and adipocytes can be delivered herein. In some instances, cells herein can be cultured ex vivo and in the culture dish environment differentiate either directly to heart muscle cells, or to bone marrow cells that can become heart muscle cells. The cultured cells can then be transplanted into a subject or a mammal, either with the biomaterial or in contact with a scaffold and other components. Myoblasts are another type of cell that lead themselves to transplantation into myocardium, however, they do not always develop into cardiomyocytes in vivo. Adult stem cells are yet another species of cell that can be part of a composition herein. Adult stem cells are thought to work by generating other stem cells (for example those appropriate to myocardium) in a new site, or they differentiate directly to a cardiomyocyte in vivo. They may also differentiate into other lineages after introduction to organs, such as the heart. In another instance, the mesenchymal stem cells are administered with activating cytokines. Subpopulations of mesenchymal cells have been shown to differentiate toward myogenic cell lines when exposed to cytokines in vitro. In some instances, cells growing with the biomaterial herein can have increased amounts of actinin, connexin43 or pan-cadherin. Cells growing with the biomaterial may have increased survivability compared to cells growing with collagen alone. Also, cells growing with biomaterial herein may have increased cell attachment to the biomaterial compared to cells growing with collagen to collagen.

The biomaterial (e.g., ECM) can be used in a cell therapy to deliver cells into the damaged tissue of target such as infarct wall following a myocardial infarction. The following list includes some of the cells that may be used in a cell therapy to be delivered to a subject with a condition (e.g., cardiovascular condition) separately or concurrently with a biomaterial injection: a human embryonic stem cell, a fetal cardiomyocyte, a myofibroblast, a mesenchymal stem cell, a endothelial cell, a amniotic mesenchymal stem cell, an autotransplanted expanded cardiomyocyte, an adipocyte, a totipotent cell, a pluripotent cell, an induced pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoietic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogeneic cell, an allogeneic cell, an adult stem cell, a post-natal stem cell, and a cardiomyocyte generated by transdifferentiation. As noted herein, differentiated cells may be used in a cell therapy are provided herein. Examples of differentiated cells include cardiomyocytes, cardiac myoblasts, and other cardiac cells described herein or known in the art. Such cells may be obtained by isolating the cells from an organ (e.g., heart) of an animal or person. Such cells may also be obtained by differentiating ES cells, iPS cells, adult stem cells, or other progenitor cells (for example cardiomyocyte progenitor cells). Methods of differentiation are known in the art. Such cells may also be obtained by transdifferentiation of cells of a different cell type altogether (e.g., bone marrow cells).

In some aspects, methods of treating a subject with a condition (e.g., cardiovascular condition) described herein can comprise steps of injecting a biomaterial (e.g., ECM) and delivering a reporter agent. The types of reporter agent can include imaging enhancers, contrast agents (e.g., angiographic contrast agents), bioluminescence agents, nano particles, and microbeads. The reporter agent can be delivered concurrently with the biomaterial in a composition, such that the delivery of the biomaterial can be monitored to ensure in situ delivery. The biomaterial may comprise the reporter agent. The reporter agent can be delivered separately with the biomaterial in a separate composition. A reporter agent can be used to enhance the contrast of structures or fluids within a subject body in medical imaging. It may be used to enhance the visibility of blood vessels and the gastrointestinal tract. Methods or treating a subject as described herein can further comprise conducting a diagnostic imaging procedure concurrently or separately from injecting to the subject a biomaterial. In some cases, the diagnostic imaging procedure can be conducted prior to the injection of the biomaterial to the subject. In some instances, the diagnostic imaging procedure can be conducted less than 1, 2, 3 weeks after the condition (e.g., cardiovascular condition, myocardial infarction). One or more types of medical imaging technology that can be used include, but are not limited to: angiography, magnetic resonance imaging, x-ray, radiography, photoacoustic imaging, thermography, ultrasound, echocardiography, positron emission tomography, and x-ray computed tomography.

Methods described herein provide a treatment to a subject with a condition (e.g., cardiovascular condition), comprising the steps of: delivering to the subject a biomaterial and delivering to the subject a therapeutic or therapeutic treatment. In some instances, one or more therapeutics or therapeutic treatments can be delivered to the subject with a condition separately or concurrently. The therapeutics can be therapeutic agents or therapeutic devices. In some instances, a biomaterial can be delivered first, followed by the delivery of one or more therapeutic agents separately. In some cases, a biomaterial can be delivered after the delivery of one or more therapeutic agents. In some instances, a biomaterial can be delivered first, followed by the delivery of one or more therapeutic devices (e.g., cardiac therapeutic devices). In the cases when a subject with a condition is treated with both therapeutic agents and therapeutic devices, a biomaterial may be delivered simultaneously with both therapeutic agents and therapeutic devices; or may be delivered separately from the concurrent delivery of therapeutic agents and therapeutic devices. In some cases, a subject with a condition may be under a therapeutic treatment continuously, and treated with a biomaterial at one or more specific time points. A subject with a condition may be treated with one or more therapeutic treatments and one or more biomaterial treatments.

As described herein, a subject with a condition (e.g., cardiovascular condition) can be treated by injecting a biomaterial (e.g., ECM) to the subject. The subject may also be delivered one or more therapeutics, such as therapeutic agents, or therapeutic devices (e.g., therapeutic cardiac devices). The biomaterial that is delivered and the one or more therapeutics may interact with one another prior to, concurrently, or upon delivering to the subject with a condition. In some instances, the biomaterial may transition to a gel form first in vitro, followed by seeding the cells to be delivered on the biomaterial. The cells may be mixed with the biomaterial solution, followed by transitioning to a gel form, resulting in encapsulation of the cells within the biomaterial. The combination of biomaterial and cells can be delivered together upon seeding of the cells in vitro. The biomaterial and each of the one or more therapeutics may be configured to have functions to directly or indirectly act upon the subject's condition. The biomaterial and the therapeutics may be configured to have the same or different functions. The biomaterial or the therapeutics may be configured to have one or more functions. In some instances, the one or more therapeutics, such as therapeutic agents or therapeutic devices, are configured to reduce a heart load. The therapeutic devices may be cardiac therapeutic devices. The subject can have one or more conditions, such as multiple symptoms or combinations of side effects that can be associated with one major condition. In some instances, the biomaterial and the therapeutics may treat the same or different conditions of the subject. In some instances, the biomaterial and the therapeutics may target the same tissue. In some instances, the biomaterial and the therapeutic may target different tissues. In some instances, the biomaterial and the therapeutic may target different parts of an organ (e.g., heart). In some instances, the biomaterial or the therapeutic may treat the subject systematically.

Methods are described herein for treating a subject with a condition (e.g., cardiovascular condition) comprise steps of: injecting a biomaterial (e.g., ECM) to the subject such that the condition of the subject is improved following the injection of the biomaterial. The subject can also be administered one or more therapeutics (e.g., therapeutic agents, cardiac therapeutic devices). In some instances, the biomaterial (e.g., ECM) may result in improvement of the condition in terms of cell engraftment, cell growth, or cell differentiation. Herein, ECM is prepared such that much of the bioactivity for tissue regeneration is preserved. Exemplary bioactivity of the biomaterial herein include without limitation: increase in cellularity, increase in cell survival, increase in cell engraftment, control or initiation of cell adhesion, cell migration, cell differentiation, cell maturation, cell organization, cell proliferation, cell death (apoptosis), stimulation of angiogenesis, proteolytic activity, enzymatic activity, cell motility, protein and cell modulation, activation of transcriptional events, provision for translation events, inhibition of some bioactivities, for example inhibition of coagulation, stem cell attraction, chemotaxis, and MMP or other enzyme activity.

In some instances, a biomaterial (e.g., ECM) herein promotes maturation of implanted cells. For example, immature cells implanted in a damaged myocardium can be implanted with or shortly following the delivery of ECM biomaterial as described herein, wherein the ECM biomaterial promotes maturation of the implanted cells. In some instances, a biomaterial promotes differentiation of implanted cells. For example, induced pluripotent stem (iPS) cells can be implanted with or shortly following the delivery of ECM biomaterial; and the ECM acts to promote differentiation of the iPS cells. In some cases, in vivo factors may also act on the iPS cells to promote differentiation, either independently or along with the ECM. In another example, embryonic stem (ES) cells, progenitor cells, cardiac progenitor cells, or adult stem cells are implanted along with, or following, the delivery of ECM; and the ES cells or adult stem cells are subsequently differentiated into more mature cell type. In some cases, in vivo factors may also act on the ES cells or adult stem cells to promote differentiation, either independently or along with the ECM. In some instances, the ECM can recruit endogenous cells to migrate to the injured region of the tissue, or the region where the ECM is injected to. In some instances, the recruited cells can reside, engraft, grow, and/or differentiate at the region.

In some instances, the biomaterial (e.g., ECM) may provide improvements in biomechanical support to an injured, diseased, damaged or ischemic tissue. In some instances, the biomaterial can result in reduction of injury size, partial or complete restoration of biomechanical property of the tissue, increase in matrix production, or up-regulation of tissue-specific markers. In some instances, the biomaterial can improve partially or completely on recovery of tissue function, or recovery of organ function. The tissue function or organ function can be evaluated by one or more functional measurements specifically for the type of tissue/organ that the biomaterial is intended to treat. In some instances, delivery of the biomaterial (e.g., ECM) to a subject may improve cardiac functions. Cardiac functions can be measured by ejection fraction (EF) or wall motion scores. An ejection fraction is a measure of cardiac function that measures the efficiency of output from the ventricles. The ECM can be delivered by injection or implantation.

In some instances, the biomaterial (e.g., ECM) may not trigger host immune responses. In some instances, the biomaterial does not substantially comprise any cellular antigens. In some instances, the biomaterial may trigger host immune responses. In some cases, the biomaterial is delivered to the subject prior to, concurrent with or after immunosuppressive therapy. In some cases, immunosuppressive therapy is not used.

In some instances, the biomaterial (e.g., ECM) may degrade over time in the subject body. In some instances, the biomaterial may not degrade over time in the subject body. In some instances, only part of the biomaterial may degrade over time in the subject body. In some embodiments, the biomaterial may degrade within 1, 2, 3, 4, 5, 6, 7, 8, 9, 10, 12, 13, or 14 days; 3, 4, 5, 6, 7, 8, 9, 10, 11, or 12 weeks; 4, 5, 6, 7, 8, 9, 10, 11, or 12 months; 2, 3, 4, 5, 6, 7, 8, 9, 10, 11, 12, 13, 14, 15, 16, 17, 18, 19, or 20 years. In some embodiments, the biomaterial may degrade after more than 20 years.

The delivery of the biomaterial may result in improvement of the condition in terms of aforementioned categories for greater than 5%, 10%, 15%, 20%, 25%, 30%, 35%, 40%, 45%, 50%, 55%, 60%, 65%, 70%, 75%, 80%, 85%, 90%, 95%, and 99%. The delivery of the therapeutics (e.g., one or more therapeutic agents or cardiac therapeutic device) may also result in improvement of the condition for greater than 5%, 10%, 15%, 20%, 25%, 30%, 35%, 40%, 45%, 50%, 55%, 60%, 65%, 70%, 75%, 80%, 85%, 90%, 95%, and 99%. In preferred embodiments, the condition is improved for greater than 50%. In some instances, the biomaterial and the therapeutic (e.g., one or more therapeutic agents, cardiac therapeutic device) may act upon the condition synergistically. In some instances, the improvement of the condition after the delivery of both the biomaterial and the therapeutic is greater than the improvement from treating the subject with the biomaterial alone or with the therapeutic (e.g., one or more therapeutic agents, cardiac therapeutic device) alone.

Methods herein can comprise delivering the biomaterial as a wound repair device. For example, after cardiac ablation, the biomaterial can be delivered in situ to improve healing. The healing can be improved by recruiting the progenitor cells to the injury site, regenerating new tissues, increasing tissue mechanical properties or providing structures for tissue growth guidance.

In some embodiments, the biomaterial (e.g., ECM) may be molded into a particular shape and then transplanted or introduced into a subject with a condition. The biomaterial may be molded into a supportive matrix of a desired shape, e.g., in the shape of a catheter, a stent, a graft, a patch or the like, which may be suitable for transplantation or introduction into a desired area in a subject (e.g., myocardium). Cells (e.g., seed cells) may optionally be planted or cultured on the biomaterial. A patch, graft or gel product or a similar transplantation construct may be prepared. The transplantation construct may be transplanted or introduced into a damaged area in a subject. In some embodiments, the transplantation (or introduction) promotes cell growth or survival in the area or otherwise populates the damaged area with cells; often, the cell or tissue growth occurs directly on the ECM biomaterial.

Methods are described herein of preparing an injectable biomaterial comprising decellularized ECM derived from tissue. The biomaterial may comprise: decellularized extracellular matrix derived from a tissue (e.g., cardiac tissue or skeletal muscle tissue). The ECM can be miscible in water, thereby forming a solution. The viscosity of the biomaterial can change under one or more changes in temperature, pressure, pH, presence of cross-linker, radiation, or the protein composition of the biomaterial. The viscosity of the biomaterial can increase such that the biomaterial eventually transitions to a gel form. The biomaterial can gel and be combined with cells, peptides, proteins, DNA, drugs, nutrients, survival promoting additives, proteoglycans, and/or glycosaminolycans, or combined and/or crosslinked with a synthetic polymer for further use.

The viscosity of the biomaterial can increase when warmed above room temperature including physiological temperatures approaching about 37° C. In some instances, the solution is a liquid solution at a temperature less than 25, 20, 15, 10, 5, or 0° C. In some instances, the solution is a gel at a temperature of more than 20, 30, 35, or 37° C. In some instances, the solution is a liquid when water is mixed with the biomaterial and the biomaterial is delivered in vivo. In some instances, the ECM biomaterial can be configured to gel at body temperature (for example 37° C. or greater). In some instances, the ECM biomaterial is configured to gel at greater than 20, 25, 30, or 35° C. According to one non-limiting embodiment, the ECM biomaterial is an injectable solution at room temperature and other temperatures below 35° C. In another non-limiting embodiment the gel can be injected at body temperature of 37° C. or near body temperature, but gels more rapidly at increasing temperatures. In some instances, a gel forms in less than 5 minutes at physiological temperature of 37° C. In some instances, a gel forms in less than 10 minutes at physiological temperature of 37° C. In some instances, a gel forms in less than 15 minutes at physiological temperature of 37° C. In some instances, a gel forms in less than 30 minutes at physiological temperature of 37° C. In some instances, a gel forms in less than 45 minutes at physiological temperature of 37° C. In some instances, a gel forms in less than 1 hour at physiological temperature of 37° C. In some instances, a gel forms in less than 5 minute at physiological temperature of 37° C.

In some instances, a biomaterial can transition to a gel forms in vivo or ex vivo. In some cases, a biomaterial may transition to gel form in less than 5 minutes in vivo. In some instances, a gel forms in less than 10 minutes in vivo. In some instances, a gel forms in less than 15 minutes in vivo. In some instances, a gel forms in less than 30 minutes in vivo. In some instances, a gel forms in less than 45 minutes in vivo. In some instances, a gel forms in less than 1 hour in vivo. In some instances, a gel can form only when the liquid biomaterial (e.g., ECM) is in direct contact in vivo with the tissue that the liquid biomaterial is delivered into.

In some instances, a biomaterial described herein may transition to gel form by forming a cross-link. An un-gelled or partially gelled biomaterial can transition to gel form when mixing with a crosslinker. The cross-links can be covalent bonds or ionic bonds. Cross-linking can also be induced in materials that are normally thermoplastic through exposure to a radiation source, such as electron beam exposure, gamma-radiation, or UV light. Cross-links are the characteristic property of thermosetting plastic materials. In some cases, cross-linking is irreversible, and the resulting thermosetting material will degrade or burn if heated, without melting. In some cases, though, if the cross-link bonds are sufficiently different, chemically, from the bonds forming the polymers, the process can be reversed. In some instances, a biomaterial can cross-link with an oxidizer such as hydrogen peroxide. Non-limiting examples of cross-linkers include glutaraldehye, formaldehyde, transglutaminase, or bis-amine reactive molecules. In some instances, the cross-linkers include, but not limited to: common collagen crosslinkers, HA crosslinkers, or other protein cross-linkers with altered degradation and mechanical properties.

A biomaterial described herein, such as ECM, can transition to gel form when the pH is at a physiological pH. The biomaterial can be in liquid form in acidic pH, and transition to gel form when a basic solution or reagent is added to bring the pH toward a neutral or physiological pH. The biomaterial can be in liquid for with 0.5 M, 0.1 M, or 0.01 M acetic acid or 0.1 M HCl. In some instances, a buffer can be added to the liquid biomaterial to initiate gelation. The biomaterial can be in liquid form in pH 1, 2, 3, 4, 5, or 6; less than 6, less than 5, less than 4, less than 3, or less than 2. The pH of the biomaterial solution can be brought toward a pH of about 6 to about 9, such that the biomaterial can transition to gel form. In some instances, the pH is brought to a physiological pH of about 7 or 7.4. In some instances, the biomaterial can transition to gel form under the pH in vivo.

In some instances the biomaterial described can transition to form a gel when the temperature is raised to physiologically relevant temperatures. The material can be in liquid form at room temperature or below, and transition into a gel when the temperature is increased. The biomaterial can be in liquid form at temperatures less than 4° C., 10° C., 25° C. or 30° C. The biomaterial can transition to a gel form when the temperature is increased to greater than 32° C., 37° C., or 40° C. In some instances, the biomaterial can transition to gel form under the temperature in vivo.

Described herein are methods and related compositions for use to treat a subject with a condition (e.g., cardiovascular condition). The subject with a condition can be treated by injecting a biomaterial (e.g., ECM) to the subject in situ. The methods can further comprise delivering a therapeutic treatment to the subject. In some instances, the biomaterial can transition to gel form after delivery of the biomaterial. The gelation of the biomaterial may happen before or after delivery of a therapeutic treatment that is configured to treat the subject with a condition. The gelation of the biomaterial can happen simultaneously to the delivery of the therapeutic treatment. The gelation may occur on a therapeutic device prior to the delivery of both the biomaterial and the therapeutic device to the subject. The gelation may occur on a therapeutic device while both the biomaterial and the therapeutic device are being delivered together to the subject. The gelation may occur only when in contact with a tissue in vivo.

In some instances, a biomaterial that can be injected into a subject with a condition can be an extracellular matrix material derived from a biological tissue from a mammal. The biological tissue suitable to derive extracellular matrix biomaterial to treat a subject with a condition can be a soft tissue or a hard tissue. Four categories of biological tissues that can derive ECM biomaterial include: an epithelial tissue, a connective tissue, a muscle tissue, or a nerve tissue. Epithelial tissue is the linings or covers of all body surfaces such as linings of a heart tissue or a skeletal muscle tissue. Non-limiting examples of epithelial tissues include skin, epithelium, dermis, mucosa, and serosa. Connective tissue is a kind of biological tissue that supports, connects, or separates different types of tissues and organs of the body. Non-limiting examples of connective tissues include vascular tissue such as arteries, veins and capillaries; blood and its constituents such as red blood cells, platelets, white blood cells; lymph, fat or adipose tissue, fiber, cartilage or fibrocartilage, ligament, tendon, bone, teeth, omentum, submucosa, peritoneum, mesentery, meniscus, conjunctiva, duramater, umbilical cord, scar tissue and bone marrow. Muscle tissues can be divided into three categories including smooth muscle, cardiac muscle, or skeletal muscle. Non-limiting examples of muscle tissue include myocardium (or cardiac tissue), skeletal muscle tissue, intestinal wall tissue, stomach tissue, and colon tissue. Nervous tissue is the main component of the nervous system including brain, spinal cord, and nerves. It is composed of neurons, and the neuroglia cells, which assist propagation of the nerve impulse as well as provide nutrients to the neuron.

In certain embodiments, the ECM biomaterial can be derived from different body organs of a mammal. The types of body organ that can be used to derive ECM can be brain, heart, lung, vessels, liver, esophagus, pancreas, stomach, large intestine, small intestine, colon, rectum, kidney, bladder, uterus, ovary, mammary gland, bone marrow, spleen, thyroid, pharynx, trachea, bronchi, diaphragm, bone, cartilage and tendon. The EXCM can be derived from skeletal muscle tissue. In some instances, the ECM biomaterial can be derived from a part of an organ or a whole organ.

In some instances, the ECM biomaterial that is injected to a subject with a condition can be derived from cardiac tissue. The cardiac tissue sample can be isolated from a mammal such as a non-primate (e.g., cows, pigs, horses, cats, dogs, rats, etc.) or a primate (e.g., monkey and human), or from an avian source (e.g., chicken, duck, etc.). For human therapy, there are many potential sources for the heart extracellular matrix material: human heart (including autologous, allogeneic, or cadaveric), porcine heart, bovine heart, goat heart, mouse heart, rat heart, rabbit heart, chicken heart, and other animal sources. In some instances, the ECM is sourced from multiple animals (e.g., multiple pigs) or multiple animal species (e.g., pigs plus a different species, such as rabbit). Unlike total heart transplantation, one donor heart could be used to treat many people. Non-human animals would be a source of heart extracellular matrix without the need for human donors. As a research reagent, non-human animal sources (porcine heart, bovine heart, goat heart, mouse heart, rat heart, rabbit heart, chicken heart, etc) can be utilized. In some instances, the cardiac extracellular matrix is derived from native tissue from embryonic or fetal sources from tissues as described herein without additional limitations. In some instances, the cardiac ECM can be derived from animals of any age. In some instances, the cardiac ECM can be derived from fetal, neonatal, immature and mature hearts.

In some instances, heart or cardiac ECM biomaterial as described herein is derived from myocardial tissue. In some instances, a biomaterial is derived from ventricular tissue. In some instances, a biomaterial is derived from left ventricular tissue. In some instances, a biomaterial can be derived from left ventricular and right ventricular tissue. In some instances, a biomaterial can be derived from autologous pericardial tissue, which may be obtained non-invasively. In other instances, heart or cardiac ECM material as described herein is derived from pericardial tissue. In some instances, ECM biomaterial herein mimics the extracellular matrix (ECM) of the tissue of injury, for example, ECM that may have been damaged by the infarction. The ECM biomaterial can provide complex, myocardial specific ECM cues, which promote repair.

In some instances, the heart tissue may be separated and divided, for example with knives and other blades, meat grinder, blender, machine that processes food, or separators. The starting muscle or organ may be cut, or processed into smaller pieces as a step to create the biomaterial.

In an aspect, a biomaterial that can be used to treat a subject with a condition can be derived from biological tissues by methods provided herein, the method comprising: decellularizing tissues; lyophilizing the decellularized tissues; digesting the lyophilized tissue with an enzyme in a first liquid; lyophilizing the digested tissue; and manufacturing a biomaterial by incorporating the lyophilized digested tissue with a second liquid. In some cases, incorporating in a liquid comprises solubilizing. In some cases, the method comprises many, but not all, of said steps. For example, the method may comprise decellularizing a tissue, digesting the tissue with an enzyme in a first liquid, and incorporating the digested tissue with a second liquid. In some cases, the method does not comprise decellularizing cardiac tissue. In some cases, the method does not comprise incorporating digested tissue with a second liquid. In some instances, the decellularized, digested and lyophilized ECM can be ground up to form a powder ECM.

In some instances, one heart is decellularized in a method herein. In some instances, two or more hearts are decellularized in a method herein. In some instances, the heart tissue is obtained from multiple animals (e.g., multiple pigs) or multiple animal species (e.g., pigs plus a different species, such as rabbit). Decellularization procedures for the cardiac tissue sample are done using one or more physical, chemical and/or biological techniques. The heart is first decellularized, leaving only the extracellular matrix and/or extracellular proteins and/or polysaccharides. In some instances, the decellularizing is carried out by using a sodium dodecyl sulfate (SDS) solution. In some instances, the enzyme is a digestive enzyme, such as pepsin, papain, trypsin, chymotrypsin, collagenase, hyaluronidase, pronase or combination thereof. In some instances, the first liquid is phosphate buffered saline (PBS), saline, or other buffered solution. In some instances, the second liquid is water, for example, sterile water, and/or deionized water, or the second liquid can be saline. In an instance, a biomaterial herein demonstrates a lack of nuclei, DNA or RNA after decellularization, when evaluated pathologically. In some instances, antibiotics or other bioburden reducing agents may be in the liquids.

In some instances, the source tissue will be treated with materials to reduce the bioburden from outside contamination. The tissue or organ may be decontaminated using agents such as, but not limited to, peracetic acid, chlorhexadine gluconate, ammonia, alcohols, aldehydes, oxidizing agents. In some instances, anti-fungal or fungicides can be used.

In one embodiment, an ECM biomaterial that can be used to treat a subject with a condition can be manufactured by methods as described herein comprising lyophilizing, grounding up, and selecting an enzyme to digest the tissue (e.g., cardiac tissue or skeletal muscle tissue). In some instances, the ECM biomaterial is ground up using a mortar and pestle, through a sonication process, or a mill. The material may also be put through a mesh in order to limit the size of the milled material. The material may be put through a 20 mesh, 40 mesh, 60 mesh or 100 mesh, or another size mesh. The material also may not be put through a mesh. In some instances, the enzyme is selected based on the desired temperature of gelation of the biomaterial when delivered in vivo. In some instances, the biomaterial transitions to a gel form within 30, 20, 10, 5, 1 or less minutes after delivery to in vivo tissue. In some instances, the tissue is digested with pepsin at a low pH, or other matrix degrading enzymes such as matrix metalloproteinases. In some instances, the biomaterial further comprises pepsin. In some instances, the biomaterial further comprises a digestive enzyme, for example, trypsin, chymotrypsin, papain, or a combination thereof. In some instances, the biomaterial further comprises a plurality of digestive enzymes as described herein. A digestive enzyme or enzymes for the composition here can be selected based on the peptide bonds that are cleaved by the enzyme or enzymes. In some embodiments, the biomaterial does not need to be neutralized after digestion. In some embodiments, the tissue is digested in a low pH, followed by neutralizing the digestive solution to a higher pH. For example, the higher pH that the digestive solution can be brought up to in order to stop the digestion may be about 5, 5.25, 5.5, 5.75, 6, 6.25, 6.5, 6.75, 7, 7.25, 7.5, 7.75, 8, 8.25, 8.5, 8.75 or 9. In some embodiments, the pH of the digestive solution can be brought up to higher than 9 to stop the digestion process. In some embodiments, the digestive solution will have salts added to it. In some embodiments, the digestive solution will have PBS added.

In some instances, the digested material may be filtered through a mesh or screen in order to exclude the size of any non-digested material. For example the liquid may be pushed through a syringe into a needle of at least 20 G, 22 G, 27 G, 30 G or other needle gauge. In some embodiments, the material is put through a screen or other apparatus with at least 20 mesh, 40 mesh, 60 mesh, 100 mesh or another size mesh. The material may not be put through any screening or filtering process.

Herein, the biomaterial comprises a matrix that is substantially decellularized. In some instances, a decellularized matrix comprises no native cells. In some instances, a decellularized matrix comprises less than 1%, 2%, 3%, 4%, 5%, 6%, 7%, 8%, 9% or 10% cellular components by weight.

In some instances, a biomaterial herein can comprise a fractionated decellularized extracellular matrix derived from a tissue (e.g., cardiac tissue or skeletal muscle tissue). For example, a biomaterial (e.g., ECM) herein comprises an extracellular matrix component (e.g., collagen fragment) with a molecular weight of less than 300 kDa, less than 200 kDa, less than 100 kDa, less than 50 kDa, or less than 20 kDa. For another example, a biomaterial herein comprises nonaqueous matrix component with a molecular weight of less than 300 kDa, less than 200 kDa, less than 100 kDa, less than 50 kDa, or less than 20 kDa. For yet another example, a biomaterial herein comprises matrix component with a molecular weight in a range with an upper limit of 300 kDa, 200 kDa, 100 kDa, 50 kDa, or 20 kDa and a lower limit of 0.5 kDa, 1 kDa, 2 kDa, 5 kDa, 10 kDa or 20 kDa.

In some instances, biomaterials provided herein that can be used to treat a subject with a condition comprise a variety of ECM proteins, after decellularization. Thus, the decellularized ECM from cardiac tissue or skeletal muscle tissue may comprise a complex combination of proteins and proteoglycans. In some instances, a biomaterial that can be injected to a subject with a condition comprises substantially all the constituents of ECM (e.g., cardiac ECM or skeletal muscle ECM). In some instances, a biomaterial comprises substantially all the constituents of the ECM at similar ratios found in the tissue it is derived from in vivo. The ECM proteins, glycoproteins, and proteoglycans may include, without limitation: collagen types I, III, IV, V, and VI, elastin, fibrinogen, lumican, perlecan, fibulin, and/or laminin. In some instances, a biomaterial as described herein comprises 90%, 80%, 70%, 60%, 50% or more of all the constituents of ECM at similar ratios found in the tissue it is derived from in vivo. In some cases, an ECM mimetic is used. For example, artificial ECM can be produced by combining either naturally-occurring or artificially-produced individual components of ECM such that the ratios of the individual components mimic the ratios found in naturally-occurring ECM.

ECM includes interstitial matrix and the basement membrane materials. In some instances, a biomaterial comprising ECM is composed of an interlocking mesh of fibrous proteins and glycosaminoglycans (GAGs). GAGs are carbohydrate polymers and are usually attached to extracellular matrix proteins to form proteoglycans. Exemplary ECM fibers that may be included in a biomaterial herein include, without limitation, perlecan, agrin, and collagen of all types including: fibrillar (Type I,II,III,V,XI); facit (Type IX,XII,XIV); short chain (Type VIII,X), basement membrane (Type IV), and other (Type VI,VII, XIII).

In some instances, the biomaterial herein can be provided in an injectable form. A decellularized matrix powder may be solubilized, digested, partially digested, turned into a suspension or partial suspension or otherwise incorporated in a liquid. In some instances, the incorporated product comprises glycosaminoglycans (GAG). For example, the biomaterial may comprise a glycosaminoglycan (GAG) content of at least about 1, 2, 3, 4, 5, 8, 10, 15, 20, 25, 30, 40, 50 μg per mg of lyophilized matrix or higher. In some instances, the ECM biomaterial comprises glycosaminoglycan content of at least 5, 10, or 15 μg per mg of the lyophilized composition. In another instance, a biomaterial herein comprises glycosaminoglycan content of between about 15 to 25 μg per mg of the lyophilized matrix. In some instances, the incorporated product comprises collagen. For example, the biomaterial may comprise a collagen content of at least 1, 2, 3, 4, 5, 8, 10, 15, 20, 25, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, or 700 μg per mg of lyophilized matrix or higher.

In some instances, the biomaterial herein can be provided in a lyophilized form, so that sterile water or PBS or other liquid can be added and used to resuspend material to be injected or used. The digested liquid can be frozen and lyophilized leaving behind a lyophilized, dried powder, cake or other form of the material. In some instances, water, sterile water, deionized water can be added to the lyophilized form and then mechanically agitated in order to resuspend the material. In one embodiment, PBS can be added to the lyophilized form and then mechanically agitated such that the material is reconstituted in the PBS. In other instances, another liquid can be used to reconstitute the lyophilized material.

The biomaterial may also be sterilized using any conventional methods that are well known in the art. Non-limiting examples include UV, EO gas and/or autoclaving with steam or heat sterilization.

Biomaterials described herein can comprise a number of factors or cues in vivo, including, but not limited to: factors that promote neovascularization such as VEGF and bFGF; factors that promote cell infiltration such as SDF; factors that alter the immune response; factors that alter the inflammatory response such as IL-10; factors that promote survival of endogenous cardiomyocytes and cardiac cells; and factors that prevent apoptosis of endogenous cardiomyocytes and cardiac cells.

In another aspect disclosed herein, a subject with a condition can be treated with a biomaterial (e.g., ECM) with a pore size of about 30 to 40 microns, wherein the material is biocompatible with the tissue (e.g., cardiac tissue) that can be treated, and wherein the material is injectable through a catheter with an inner diameter of 25 G or smaller. The pore size in between 30 to 40 may be important for cell infiltration upon delivery to the subject in vivo. The biomaterial (e.g., ECM) may have a pore size of about 5, 10, 15, 20 or 25 microns. In some instances, a biomaterial (e.g., ECM) can be used to treat a subject with a condition comprises a material with a pore size of less than 50 microns. In some instances, the biomaterial (e.g., ECM) has a pore size of about 50 to 100 microns.

In some aspects of the method described herein, a subject can be treated by injecting a biomaterial (e.g., ECM) to the subject, wherein the biomaterial is a liquid that has a viscosity allowing delivery of the biomaterial. In some instances, the biomaterial is deliverable through a needle or catheter. Viscosity measures the resistance of a liquid that can be deformed by shear stress or tensile stress. In some instances, the biomaterial is a thick liquid that has a high deliverable viscosity. In some instances, the biomaterial is a thin liquid that has a low deliverable viscosity. In some instances, the biomaterial has a viscosity that is greater than water. In some instances, the biomaterial has a viscosity that is less than water. The viscosity of the biomaterial may be greater than 1, 10, 100, 500, 1000, 1500, 2000, 3000, 4000, 5000, 6000, 7000, 8000, 9000, 10000, 15000, 20000, 25000, 30000, 35000, 40000, 45000, 50000, 60000, 70000, 80000, 90000, 100000, 150000, 200000 or 250000 mPa·s. The viscosity of the biomaterial may be less than 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2 or 0.1 mPa·s. In some instances, the viscosity of the biomaterial can change during delivery. For example, the biomaterial can become thicker during injection while still remaining deliverable. The viscosity may change according to the concentration of the liquid ECM composition. In some cases, the viscosity of a liquid ECM may depend on the composition of the ECM. The ECM can be a shear thinning liquid.

The methods and compositions described herein comprise a biomaterial (e.g., ECM) that can be injected directly into a subject and thereby can be used as a material therapy. For example, the biomaterial can be in a liquid solution comprising a extracellular matrix that is derived from cardiac tissue or skeletal muscle tissue. The solution can be neutralized and brought up to the appropriate concentration using PBS, saline or other buffers. The solution can be solubilized in water. The ECM may be solubilized and delivered in a concentration that is about, at least about, less than about 1, 2, 3, 4, 5, 6, 7, 8, 9, 10, 11, 12, 13, 14, 15, 16, 17, 18, 19 or 20 mg/ml. The ECM may be solubilized and delivered in a concentration that is between about 1-20, 1-10, 2-8, or 4-8 mg/ml. The solution comprising the cardiac or skeletal muscle ECM can then be injected through a small diameter needle into the myocardium. Such solution then may form a gel in vivo.

As described herein, a biomaterial (e.g., ECM) can be delivered to a subject with a condition. The biomaterial herein can be solubilized and delivered as a liquid composition. The liquid composition herein can comprise a decellularized ECM derived from cardiac tissue or skeletal muscle tissue, and another component or components. In some instances, the amount of ECM in the total liquid composition is greater than 90% or 95% of the liquid composition by weight. In some embodiments, the ECM in the total liquid composition is greater than 1%, 5%, 10, %, 20%, 30%, 40%, 50%, 60%, 70%, or 80% of the composition by weight. In another instance, a liquid composition herein comprises fractions of cardiac tissue or skeletal muscle ECM biomaterial with molecular weight bands below about 20 kDa. In some instances, the ECM biomaterial comprises fractions of cardiac tissue or skeletal muscle ECM with molecular weight bands that are around 10, 20, 30, 40, 50, 60, 70, 80, 90, or 100 kDa. In some instances, the ECM biomaterial comprises fractions of cardiac tissue or skeletal muscle ECM with molecular weight bands that are around 90 kDa.

In some instances, a subject with a condition can be treated by injecting to the subject a biomaterial (e.g., ECM). In some instances, the ECM can comprise collagen, elastin, GAG or a combination thereof. In some instances, the ECM biomaterial can be solubilized and delivered as a liquid composition. In some instances, the ECM that is derived from cardiac tissue or skeletal muscle tissue can comprise collagen that is high enough to allow the liquid composition to transition to gel form upon delivery to the subject in vivo, without further addition of any gelling agent, such as methylcellulose. In some instances, the cardiac or skeletal muscle ECM can have a collagen or elastin content that allows delivery through a needle or a catheter, followed by gelation upon delivery in vivo. The concentration of the collagen in said liquid composition can be higher than 100, 200, 300, 400, 500, 600, 700, 800, 900, 1000, 1100, 1200, 1300, 1400, 1500, 1600, 1700, 1800, 1900 or 2000 μg/ml. In preferred instances, the collagen concentration in said liquid composition is about 1000-2000 μg/ml. The concentration of the elastin in said liquid composition can be about 1, 2, 3, 4, 5, 6, 7, 8, 9, 10, 15, 18, 20, 25, 30, 35, 40, 45, 50, 60, 70, 80, 90, 100, 150, 200, 250, 300, 350, 400, 450, 500, 550, 600, 700, 800, 900 or 1000 μg/ml. The concentration of the GAG in said liquid composition can be about 5, 10, 15, 20, 25, 30, 35, 40, 45, 50, 55, 60, 65, 70, 75, 80, 85, 90, 95, 100, 125, 150, 200, 250, 300, 400, 500, 600, 700, 800, 900 or 1000 μg/ml.

Described herein are methods and compositions to treat a subject with a cardiovascular condition using a biomaterial that comprises cardiac tissue or skeletal muscle ECM for injection into a damaged cardiac tissue in the subject. In some instances described herein, an injectable gel form of a biomaterial composition derived from native heart extracellular matrix (ECM) is provided. The gel can also be used alone to recruit cells into the injured tissue or as a drug delivery vehicle. The gel can also be used to support injured tissue or change the mechanical properties. Another use of the invention is as a non-destructive conduction block to treat arrhythmias. In other instances, the biomaterial comprising a decellularized cardiac tissue or skeletal muscle ECM is configured to be in a form such as, for example, an injectable liquid form, a paste, a coating, a strand, a strip, a spray, am aerosol, a cream, a patch, an emulsion, a viscous liquid, a gel, fragments, particles, microbeads, nanobeads, a powder or a scaffold. In some instances, a biomaterial herein is a coating. The coating can be used to coat, for example without limitation, synthetic or other biologic scaffolds/materials, or implants. In some instances, a coating is texturized or patterned. In some instances, a method of making a coating includes adsorption or chemical linking. A thin gel or adsorbed coating can be formed using a solution form of the biomaterial.

In some instances, the biomaterial can comprise a biological group that can act as an adhesive or anchor where the biomaterial is delivered. In an instance, a biomaterial here can be a bioadhesive, for example, for wound repair. In some instances, a biomaterial herein can be configured as a cell adherent. For example, the biomaterial herein can be coating or mixed with on a therapeutic device that does or does not comprise cells. For example, the biomaterial herein can be a coating for a synthetic polymer vascular graft. In some instances, the biomaterial is anti-bacterial or anti-bacterial agents could be included.

In some instances, a biomaterial is injectable. An injectable biomaterial can be, without limitation, a powder, liquid, particles, fragments, gel, or emulsion. The injectable biomaterial can be injected into a heart or in many instances, injected into the left ventricle, right ventricle, left atria, right atria, or valves of a heart. The biomaterial herein can recruit, for example without limitation, endothelial, smooth muscle, cardiac progenitors, myofibroblasts, stem cells, and cardiomyocytes.

In an instance, a biomaterial (e.g., ECM) can be in a form of nanofibers. In some instances, the nanofiber biomaterial can be fabricated by electrospinning. In some instances, a biomaterial described herein can be fabricated with controlled nanofiber size, shape, alignment or thickness.

Methods herein include treating a subject with a condition by delivering a biomaterial comprising an ECM. The ECM can be delivered by methods include, but are not limited to: implantations, spraying directly onto the region of injury, direct application onto the region of injury, and injection. In another instance, the biomaterial is gelled onto a shape or product to create a device coated with the biomaterial. In another instance, the biomaterial is gelled into a shape or mold and then lyophilized. In another instance this lyophilized shape can be used as a scaffold and implanted in vivo, or can be rehydrated prior to use. The ECM can be implanted by surgery, such as open-chest surgery. The ECM can be implanted with sutures or bioadhesives. The ECM can be implanted separately or concurrently with a therapeutic device or medical device, such as stents.

The biomaterial (e.g., ECM) described herein can be delivered by injection through a needle or a catheter. The type of catheter can be any catheter that is known in the medical art. Non-limiting examples of injection method include: direct injection during surgery; direct injection through chest wall; delivery through catheter into the myocardium through the endocardium; delivery through coronary vessels; delivery through a cardiac catheter with a femoral artery access, and delivery through infusion balloon catheter. The needle size can be without limitation 22 g, 23 g, 24 g, 25 g, 26 g, 27 g, 28 g, 29 g, 30 g, or smaller. The needle can be a high gauge needle, or smaller than 25 g. In an embodiment, the needle size through which the gel is injected is 27 g. Delivery can also occur through a balloon infusion catheter or other non-needle catheter. At body temperature, the solution can then form into a gel.

The catheter that is used to deliver a biomaterial to a subject described in the current invention can have a length. The catheter can have a length that is originally designed for male, female, and pediatric. The male length catheters can be about 16 inches in length, and female length catheters can range from about 6-8 inches in length. There are some instances where females prefer to use male-length catheters. Pediatric length catheters can range from about 6-12 inches in length. In some aspects, the length of the catheter can be 40 cm. In some cases, the subject can use a catheter with a length that is adjusted to the height of the subject. In some cases, the catheter can have a length that is sufficient to deliver a biomaterial to the heart of the subject. In some embodiments, the length of the catheter can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 inches. In some embodiments, the length of the catheter can be more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 inches. In some embodiments, the length of the catheter can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 inches. In some embodiments, the length of the catheter can be more than 3 inches, more than 5 inches, more than 6 inches, more than 8 inches, more than 10 inches, or more than 12 inches. In some embodiments, the length of the catheter can be 1-50, 3-20 or 6-16 inches.

Catheters can have a straight tip or a coude tip. The coude tip catheter may be used when a blockage or stricture is present, making the use of a straight catheter more difficult. The catheter can have a tip that contains an anchor to grasp onto a tissue such that the liquid can be delivered/injected more accurately. In some embodiments, the anchor can be a screw anchor.

In some instances, the catheter can have a nominal pressure. The nominal pressure can be about 1, 2, 3, 4, 5, 6, 7, 8, 9, 10, 11, 12, 13, 14, 15, 16, 17, 18, 19 or 20 atm. The nominal pressure can be more than 1, 2, 3, 4, 5, 6, 7, 8, 9, 10, 11, 12, 13, 14, 15, 16, 17, 18, 19 or 20 atm. The nomical pressure can be less than 1, 2, 3, 4, 5, 6, 7, 8, 9, 10, 11, 12, 13, 14, 15, 16, 17, 18, 19 or 20 atm.

In some instances, the catheter can have a rated burst pressure. The related burst pressure can be about 5, 6, 7, 8, 9, 10, 11, 12, 13, 14, 15, 16, 17, 18, 19, 20, 21, 22, 23, 24, 25, 26, 27, 28, 29 or 30 atm. In some embodiments, the related burst pressure can be more than 5, 6, 7, 8, 10, 11, 12, 13, 14, 15, 16, 17, 18, 19, 20, 21, 22, 23, 24, 25, 26, 27, 28, 29 or 30 atm. In some embodiments, the related burst pressure can be less than 5, 6, 7, 8, 10, 11, 12, 13, 14, 15, 16, 17, 18, 19, 20, 21, 22, 23, 24, 25, 26, 27, 28, 29 or 30 atm.

In some instances, the biomaterial can be delivered transaxillarily, transumbilically, or transabdominally via a catheter. In another aspects, a biomaterial described herein can be injected using transcoronary delivery similar to intracoronary injection of bone marrow cells. In this example, an over-the-wire angioplasty balloon can be inflated at the occlusion site, and the composition can be infused via the guide wire lumen. In an exemplary method, a biomaterial can be injected using transendocardial delivery. In this example, a NOGA guided Myostar Catheter can be used to create a three-dimensional endocardial map of electromechanical function. Using the three dimensional map, transendocardial injections of the composition can be performed at or near the site of delivery, or the site of a myocardial infarct. In some instances, the delivery of the biomaterial can be guided by other mapping system. In some instances, the delivery of the biomaterial can be monitored by in vivo imaging technologies including, but are not limited to: x-ray, x-ray computed tomography, computed axial tomography (CAT scan), electromagnetic imaging, magnetic resonance imaging, echocardiogram and positron emission tomography.

In some instances, a subject with a cardiovascular condition can be treated by injecting a biomaterial to the subject and performing a coronary artery bypass graft (CABG) surgery on the subject. In some instances, the biomaterial can be directly injected during thoracotomy. In some cases, the injection of the biomaterial can be performed concurrently with the CABG surgery. For example, the biomaterial can be infused into coronary arteries to be delivered to the ischemic region of the myocardium. The biomaterial can be configured to gel after delivery to the myocardium while remaining in a liquid form in the coronary arteries.

Methods described herein comprise delivering a biomaterial (e.g., ECM) to a subject. In some cases, the biomaterial is a liquid that can transition to gel form when a portion of the biomaterial is mixed with a cross-linker or a gelling agent. In some instances, a liquid composition comprising ECM can be injected to an infracted region of the myocardium first, followed by injection of another liquid composition comprising a cross-linker or a gelling agent, such that the ECM composition and the cross-linker composition can be mixed or in contact in vivo, such that the ECM forms a gel after contacting the cross-linker in situ of the myocardium.

A subject with a condition may be treated by delivering to the subject a biomaterial composition comprising ECM and a therapeutic such as one or more therapeutic agents. The administration or delivery routes of the biomaterial and one or more therapeutic agents may be the same or different. The one or more therapeutic agents appropriate for delivery to a subject may be delivered in any suitable manner. Such delivery may be oral and/or any other suitable delivery, such as transdermal, intravenous, intraperitoneal, intramuscular, vaginal, rectal, and subdermal. Non-limiting examples of delivery or administration routes can include: oral consumption, intravenous injection, inhalation, nasal insufflation, intraarterial injection, intramuscular injection, topical administration, subcutaneous administration, mucosal administration, endotracheal administration, pharyngeal administration, rectal administration, sublingual administration or vaginal administration. Components of a biomaterial composition, such as ECM derived from cardiac tissue or skeletal muscle tissue and one or more therapeutic agents may be delivered to the subject concurrently, such as in any manner of concurrent delivery or administration described herein and/or in U.S. Patent Application Publication No. US 2006/0089335 A1. The biomaterial and the one or more therapeutic agents may be delivered concurrently but in different delivery routes. A biomaterial such as ECM may be delivered to a subject separately from delivery of one or more therapeutic agents in different delivery routes. Non-limiting examples of different delivery route can be: delivering the biomaterial through a catheter while delivering the therapeutic agents orally.

Methods and compositions described herein that can be used to treat a subject with a condition (e.g., cardiovascular condition) may comprise delivering a biomaterial (e.g., ECM) to the subject with a condition. In some instances, the biomaterial can be delivered by injection through a needle or a catheter in one procedure. The subject with a condition can be treated by injecting the biomaterial for one or more times per procedure; for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 injections can be performed in one procedure. In some instances, more than 30 injections can be performed in a procedure to treat the subject with a condition. In some instances, each of the multiple injections in one procedure can be at the same injection site or at different injection sites; for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 different sites can be injected in one procedure. In some instances, more than 30 injection sites can be injected in a procedure to treat the subject with a condition.

In another aspect, a biomaterial (e.g., ECM) can be delivered to a subject with a condition. In some instances, the biomaterial can be injected. In some instances, the biomaterial can be delivered to the subject for one or more times. The biomaterial can be delivered to the subject in at least 1, 2, 3, 4, 5, 6, 7, 8, 9, 10, 11, 12, 13, 14, 15 injections. The biomaterial can be delivered to the subject in more than 10 injections. The biomaterial can be delivered to the subject in 15 injections. The volume of each delivery can be the same or different. The volume of each delivery (e.g., injection) can be about, at least about, less than about 10, 20, 25, 30, 40, 50, 60, 70, 75, 80, 85, 90, 95, 100, 110, 120, 130, 140, 150, 160, 170, 180, 190, 200, 210, 220, 230, 240, 250, 300, 400, 500, 600, 700, 800, 1000, 1500, 2000, 3000, 4000, 5000 μL with an injection volume. The volume of each delivery (e.g., injection) can be less than 1 ml. The volume of each delivery (e.g., injection) can be more than 5 ml. In some instances, the total volume of delivery in each procedure can be about 1, 2, 3, 4, 5, 6, 7, 8, 9, 10, 11, 12, 13, 14, 15, 16, 17, 18, 19, 20, 21, 22, 23, 24 or 25 ml. In some instances, the total volume of delivery can be about 1-10 ml. In preferred embodiments, the total volume of delivery can be about 3-8 ml. The total volume delivered should be in a range that is not detrimental to the cardiac function and still demonstrates an effect.

In another aspect, a biomaterial (e.g., ECM) may be used to treat a subject with a condition in a single injection in a single procedure or treatment, a single injection in multiple procedures, multiple injections in single procedures, or multiple injections in multiple procedures. In some instances, a biomaterial can be delivered or injected to the subject in at least 1, 2, 3, 4, 5, 6, 7, 8, 9, 10, 11, 12, 13, 14, 15, 16, 17, 18, 19 or 20 procedures. In some instances, the biomaterial is delivered to the subject in more than 20 procedures. In some instances, the procedures are repeated. In some instances, the procedures are repeated every 1, 2, 3, 4, 5, 6, 7, 8, 9, 10 11 or 12 months. In some instances, the procedures are repeated every 3 months. In some instances, the procedures are repeated for up to 5 times.

The methods and compositions described herein can be used to treat a subject with a condition (e.g., cardiovascular condition). The subject can be injected with a biomaterial comprising decellularized cardiac extracellular matrix derived directly from native cardiac tissue, such that the biomaterial is used for treating defective, diseased, damaged or ischemic tissues or organs in the subject. The subject can be any subject that can be suffering from a condition, e.g., the subject may be an animal, a vertebrate animal, or a mammal, e g, a dog, a cat, a rat, a mouse, a horse, a rabbit, a guinea pig, a sheep, a goat, a bovine, a pig, and a human. In some instances, the biomaterial is delivered to a non-human animal subject. In preferred instances, the subject is a human subject.

The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 year old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subject can be greater than 40 years old. In preferred embodiments, the subject can be 30 to 75 years old. In other instances, the subject is younger than 30 years old or older than 75 years. The human subjects can include male subjects and/or female subjects.

As described herein, methods and compositions comprising a biomaterial can be used to treat a subject with a condition. The types of condition that can be treated include, but are not limited to, cardiovascular conditions, skeletal muscle condition, connective tissue condition, orthopaedic tissue condition and epithelial tissue condition. Non-limiting examples of skeletal muscle condition include: skeletal muscular injuries and muscular dystrophy. Non-limiting examples of orthopaedic tissue condition include: arthritis, meniscus injuries, ligament injuries, tendon injuries, cartilage injuries, sports injuries or degenerative joint diseases. In particular, the condition or disease that can be treated also include, but are not limited to: burns, ulcer, bone fracture diabete, arthritis asthma, trauma, wound, infection, cancer, cystitis, stricture, aortic aneurysm, hemophilia, ischemia, stroke, internal organ failure, uterine fibroid, urinary incontinence, and the like. In some instances, one or more different conditions or diseases can be treated at the same time. In particular instances, non-limiting examples of the cardiovascular condition include: myocardial infarction, ischemic heart disease, coronary heart disease, cardiomyopathy, hypertensive heart disease, heart failure, cor pulmonale, cardiac dysrhythmia, inflammatory heart disease, valvular heart disease, stroke and cerebrovascular disease, and peripheral arterial disease. In preferred embodiments, the cardiovascular condition can be acute ST-elevation myocardial infarction (STEMI). The cardiovascular condition can also be non acute ST-elevation myocardial infarction (STEMI).

In particular instances, the biomaterial can be delivered or injected in situ to treat defective, diseased, damaged, injured or ischemic tissues or organs. Non-limiting examples of organs to be treated include: heart, muscles, eye, breast, ovaries, lung, bladder, bone, cartilage, ligament, tendon, colon, rectum, stomach, bone marrow, brain, diaphragm, thyroid, blood vessels, hair, spinal cord, liver, pancreas, kidney, reproductive organs, cervix, and nerve. In some instances, one or more different organs can be treated at the same time.

Described herein is a method for treating a subject with a cardiovascular condition (e.g., myocardial infarction), comprising injecting to the subject a biomaterial that may be myocardial-specific via a catheter to promote repair in the post-MI environment. The method can further comprise delivering to the subject a cardiac therapeutic treatment. The myocardial-specific biomaterial may comprise a complex mixture of ECM proteins, peptides, and polysaccharides that are derived from left ventricular tissues. In many instances, the biomaterial composition is a liquid at room temperature and forms a porous and fibrous scaffold upon injection into the myocardium. The biomaterial composition promotes cell influx and preserves LV geometry and cardiac function when delivered in vivo to a myocardial infarct. In particular instances, the method described herein can be used to treat a subject with a cardiovascular condition including, but are not limited to: myocardial infarction, ischemic heart disease, coronary heart disease, cardiomyopathy, hypertensive heart disease, heart failure, cor pulmonale, cardiac dysrhythmia, inflammatory heart disease, valvular heart disease, stroke and cerebrovascular disease, and peripheral arterial disease.

In some instances, the cardiovascular condition of a subject can be a secondary condition. The secondary condition can be secondary to ischemia. In some instances, the secondary condition to the cardiovascular condition of a subject can be an iatrogenic cause. Iatrogenic cause in subject with the cardiovascular condition can occur, for example, with central venous line insertion, cardiac catheterization procedures, or pericardiocentesis. Non-limiting examples of iatrogenic cause can be infection, iatrogenic superior vena cava syndrome from CABG, death of cardiomyocytes via apoptosis or necrosis, or iatrogenic cardiac herniation.

The composition can be injectable into patients with an acute ST-elevation myocardial infarction (STEMI). The STEMI can be within the prior 7 to 21 days. In some cases, the patients have a non-acute ST-elevation myocardial infarction. In some cases, the patients had a reperfusion intervention (e.g. percutaneous coronary intervention devices). Patients treated by the composition may have a left ventricular ejection fraction (EF) of greater than or equal to 25% but less than or equal to 55% as measured by echocardiography during the screening period (3-5 days post index event). In some cases, the patients has a EF of 35%-45%. In some cases, the patients have an EF of less than 25% or 35%. In other cases, the patients have an EF of greater than 45% or 55%.

In some instances, patients have Thrombolysis. In Myocardial Infarction (TIMI) II flow scores before receiving an intervention. The TIMI Risk Score assesses the risk of death and ischemic events in patients. The patients can be experiencing unstable angina, or a non-ST elevation myocardial infarction. The patients can be experiencing a ST-elevation myocardial infarction. The TIMI risk score is a simple prognostication scheme that categorizes a patient's risk of death and ischemic events and provides a basis for therapeutic decision making. TIMI Score can be calculated as designating 1 point for each category as follows: Age >=65; Aspirin use in the last 7 days (patient experiences chest pain despite ASA use in past 7 days); At least 2 angina episodes within the last 24 hrs; ST changes of at least 0.5 mm on admission EKG; Elevated serum cardiac biomarkers; Known Coronary Artery Disease (CAD) (coronary stenosis >=50%); At least 3 risk factors for CAD, such as: Hypertension->140/90 or on antihypertensives, current cigarette smoker, hypercholesterolemia, diabetes mellitus, Family history of premature CAD (CAD in male first-degree relative, or father less than 55, or female first-degree relative or mother less than 65). The score interpretation can be (% risk at 14 days of: all-cause mortality, new or recurrent MI, or severe recurrent ischemia requiring urgent revascularization): Score of 0−1=4.7% risk; Score of 2=8.3% risk; Score of 3=13.2% risk; Score of 4=19.9% risk; Score of 5=26.2% risk; Score of 6-7=at least 40.9% risk. TIMI risk can estimate mortality following acute coronary syndromes. TIMI risk can also be calculated on the TIMI website under Clinical Calculators. The TIMI Grade Flow is a scoring system from 0-3 referring to levels of coronary blood flow assessed during percutaneous coronary angioplasty. TIMI 0 flow (no perfusion) can refer to the absence of any antegrade flow beyond a coronary occlusion. TIMI 1 flow (penetration without perfusion) can be faint antegrade coronary flow beyond the occlusion, with incomplete filling of the distal coronary bed. TIMI 2 flow (partial reperfusion) can be delayed or sluggish antegrade flow with complete filling of the distal territory. TIMI 3 can be normal flow which fills the distal coronary bed completely

In yet another aspect described herein, a method for repairing cardiac tissue may comprise injecting or implanting into a subject a biomaterial comprising decellularized extracellular matrix derived from cardiac tissue or skeletal muscle. In some instances, the biomaterial may be injected or implanted earlier than one month following a condition (e.g., myocardial infarction) or the biomaterial is injected or implanted earlier than two weeks following the condition (e.g., myocardial infarction). In some instances, the biomaterial is injected or implanted earlier than 1 day, 2 days, 3 days, 4 days, 5 days, 6 days, 1 week, 2 weeks, 3, weeks, 10 days, 20 days, 45 days, two months, three months, four months, 6 months or 1 year following the condition (e.g., myocardial infarction). In some instances, the biomaterial is injected or implanted after 1 year following the condition (e.g., myocardial infarction). In some instances, the biomaterial degrades within three months or within one month following injection or implantation. In some instances, the biomaterial degrades within 6 weeks, 2 months, 4 months, 6 months or 1 year following injection or implantation. In some instances, injection or implantation of said biomaterial repairs a congenital defect. In some instances, the delivery of the biomaterial occurs about 1 to 30 days after the condition (e.g., myocardial infarction). In some instances, the delivery of the biomaterial occurs at least 1 month or at least 1 year after the condition (e.g., myocardial infarction). The delivery of the biomaterial occurs about 1 to 24 hours after the condition (e.g., myocardial infarction).

In an aspect, a method for treating a subject with a cardiovascular condition comprises delivering to the subject a biomaterial comprising decellularized extracellular matrix derived from cardiac tissue or skeletal muscle. The method further comprises sterilizing the ECM prior to the delivery of the biomaterial to cardiac tissue. In some instances, the ECM is sterilized by ethylene oxide or by radiation. In some instances, the ECM is solubilized in a liquid solution, wherein the liquid solution is water, saline, or a buffer solution. In some instances, the delivery is percutaneous, for example, where the biomaterial is delivered by a transendocardial or transcoronary a catheter.

In some instances, a biomaterial (e.g., ECM) can be delivered to the heart of a subject after myocardial infarction for more than one times. In some instances, the ECM can be delivered at the same or different locations in heart each time. In some instances, the ECM is delivered to a myocardial infarct, a border zone of a myocardial infarct, within 2 cm or less from a myocardial infarct or the healthy tissue surrounding the infarct. Delivery to a border zone of a myocardial infarct has unexpected advantages as compared to the center of an infarct zone. The cell influx and tissue regeneration potential can be higher at the border zone. For example, the step of delivering the ECM can alter ventricular remodeling. In some instances, the ECM may be injected into the left ventricle, right ventricle, left atrium, or right atrium of the heart of the subject.

In other instances, injection or implantation of ECM may prevent or repair damages to cardiac tissue sustained by a subject. In some instances, the subject is also treated with a therapeutic (e.g., one or more therapeutic agents or a cardiac therapeutic device). In some instances, injection or implantation of said ECM can further prevent or repair damages to the cardiac tissue sustained by a subject that is also treated with a therapeutic. The damage can be a myocardial infarct. The repair can be an improvement of cardiac geometries, such as ventricular volume. The ventricular volume can be end systolic volume (ESV) or end diastole volume (EDV). In some instances, said repair may comprise at least 20% less change in ventricular volume 3 months after said myocardial infarction as compared to a subject with cardiac tissue damages and without injection of implantation of the ECM. In some instances, said repair may comprise at least 20% less change in ventricular volume 3 months after said myocardial infarction as compared to a subject that is also treated with a therapeutic and without injection of implantation of the ECM. In some instances, said repair comprises at least 5, 10, 15, 20, 25, 30, 35, 40, 45, 50, 55, 60, 70, 80 or 90% less change in ventricular volume 3 months after said myocardial infarction as compared to a subject with cardiac tissue damage and without injection of implantation of the ECM. In some instances, said repair comprises at least 5, 10, 15, 20, 25, 30, 35, 40, 45, 50, 55, 60, 70, 80 or 90% less change in ventricular volume 3 months after said myocardial infarction as compared to a subject treated with a therapeutic and without injection of implantation of the ECM. In some instances, said repair comprises at least 5, 10, 15, 20, 25, 30, 35, 40, 45, 50, 55, 60, 70, 80 or 90% less change in ventricular volume 3 months after said injection or implantation as compared to a subject with cardiac tissue damage and without injection of implantation of the ECM. In some instances, said repair comprises at least 5, 10, 15, 20, 25, 30, 35, 40, 45, 50, 55, 60, 70, 80 or 90% less change in ventricular volume 3 months after said injection or implantation as compared to a subject treated with a therapeutic and without injection of implantation of the ECM.

In some instances, delivering a biomaterial (e.g., ECM) to a subject after a cardiovascular condition (e.g., myocardial infarction) may improve or repair cardiac functions. In some instances, the subject is also treated with a therapeutic such as one or more therapeutic agents or a cardiac therapeutic device. Cardiac functions can be measured by ejection fraction (EF) or wall motion scores. An ejection fraction is a measure of cardiac function that measures the efficiency of output from the ventricles. The ECM can be delivered by injection or implantation. In some instances, the repair may comprise a 20% increase in ejection fraction 3 months after said myocardial infarct as compared to a subject with cardiac tissue damage and without injection of implantation of the ECM. In some instances, the repair comprises a greater than 10, 20, 25, 30, 40, 50, 60, 70, 75, 80, 90, 100 or 200% increase in ejection fraction 3 months after said myocardial infarct as compared to a subject with cardiac tissue damage and without injection of implantation of the ECM.

In some instances, delivering a biomaterial (e.g., ECM) to a subject after a cardiovascular condition (e.g., myocardial infarction) may improve wall motion score index (WMSI). In some instances, the subject can also be treated with a therapeutic such as one or more therapeutic agents or a cardiac therapeutic device. The wall motion score index is measured by a 16-segment model as recommended by the American Society for Echocardiography. Each heart is divided into 16 segments and each segment of the heart is analyzed individually. Each segment of the heart is scored on the basis of its motion and systolic thickening such as: normal or hyperkinesis=1, hypokinesis=2, akinesis=3 and dyskinesis (or aneurysmatic)=4. The WMSI is an average score of all segments. In some instances, said improvement in WMSI can be about 1 point decrease in the subject that is injected or implanted with the biomaterial compared to a subject that is not injected or implanted with the biomaterial. In some instances, said improvement in WMSI can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 points, less than 1, 2, 3 or at least 0.1, 0.5, 1 decrease in the subject that is injected or implanted with the biomaterial compared to a subject that is not injected or implanted with the biomaterial.

Methods for delivering a biomaterial are described herein. A biomaterial (e.g, ECM) can be placed in contact with a defective or absent myocardium, resulting in myocardial tissue regeneration and restoration of contractility, conductivity, or functions of the heart muscle. In some instances, a biomaterial herein may recruit endogenous cells and can coordinate the function of the newly recruited or added cells, allowing also for cell migration and proliferation within the biomaterial. As described herein, in some cases, the biomaterial can aid the repair of myocardial tissue. In some instances, such repair involves restoration of heart tissue and/or specific features of heart tissue such as striations, T-tubules, or intercalated discs.

In yet another embodiment, a biomaterial (e.g., ECM) can be injected into the infarct area, border zone, or myocardium alone or in combination with one or more therapeutic agents such that endogenous cell ingrowth, angiogenesis, and regeneration can be achieved. In yet another embodiment, ECM biomaterial can also be used as a matrix to change mechanical properties of the heart and/or prevent negative left ventricular remodeling. In yet another embodiment, the ECM can be delivered with cells for regenerating myocardium. In yet another embodiment, the ECM can be used for creating a conduction block to treat arrhythmias.

In some instances, a subject with a condition can be treated by delivering a biomaterial (e.g., ECM) to the subject, such that a symptom of the condition is improved. The symptom that is improved can be primary symptom, secondary symptom, tertiary symptom or a combination thereof. In some instances, the symptom is dyspnea.

In some instances, a subject can show remodeling in the myocardium after myocardial infarction. The increase in mechanical loads in the heart after myocardial infarction induces a unique pattern of remodeling involving the infarcted border zone and remote non-infarcted myocardium. Remodeling can result in myocyte necrosis, dilatation, hypertrophy, and formation of a discrete collagen scar. In some instances, the subject with a cardiovascular condition (e.g., myocardial infarction) can be treated by methods comprising delivering a biomaterial (e.g., ECM) to the subject such that the remodeling of the myocardium is prevented. In some instances, the subject with a cardiovascular condition can be treated by methods comprising delivering a biomaterial (e.g., ECM) to the subject such that the remodeling of the myocardium is reversed.

In some instances, a subject with a condition can be treated by injecting to the subject a biomaterial comprising ECM. The ECM described herein can be configured to be provided for therapy in an off-the-shelf manner. In an example, a biomaterial herein can be prepared to be in a dry solid form and can be stored on a shelf or in a room or unit, and then solubilized with water, saline, or another liquid solution immediately before treatment. In some instances, the biomaterial can be stored as a liquid. In some instances, the biomaterial can be stored as a frozen solid. In some instances, the biomaterial can be stored in a gel form. In some instances, the biomaterial may be lyophilized and stored at a temperature of less than 25° C., less than 0° C., less than −20° C., or less than −70° C. In some instances, the biomaterial has a shelf-life that is about 1, 2, 3, 4, 5, 6, 7, 8, 9, 10, 11 or 12 months; 1.5, 2, 3, 4 or 5 years. In preferred instances, the biomaterial can be stored for about 6 months at a temperature of less than 25° C.

### EXAMPLES

The disclosure is further illustrated by the following examples, which are not to be construed in any way as imposing limitations upon the scope thereof. It is apparent for skilled artisans that various modifications and changes are possible and are contemplated within the scope of the current invention.

### Example 1

The objective of this study is to examine the use of a gel as a growth platform for cell adhesion, growth, maturation, and delivery in vivo. It is provided that a gel composed of native heart extracellular matrix tissue can aid in cardiac tissue regeneration by promoting cell survival.

Female Sprague Dawley rats are euthanized and their hearts harvested. The aorta and pulmonary artery are transected. The aorta is cannulized and attached to a modified Langendorff setup. The heart is decellularized by perfusing a 1% sodium dodecyl sulfate (SDS) and PBS solution for 24 hours and then a 1% triton/PBS solution for 30 minutes through the coronary vessels of the heart. Once the decellularization is completed, the heart is rinsed with water. Decellularized hearts are then lyophilized, rehydrated, pulverized, and lyophilized again to form a dry powder. Frozen hearts are rehydrated with water and then immersed in liquid nitrogen. Once frozen, hearts are systematically crushed within a ball and cup apparatus at 70 psi for 10 seconds. Pulverized heart particulates are then freeze dried. Once dry, lyophilized heart tissue is combined with 1% pepsin and amalgamated with 0.01M HCl to a concentration of 10 mg/mL. Solution is stirred at room temperature for 48 hours to allow for solubilization of the extracellular matrix tissue. After 48 hours, the HCl solution is aliquoted into Eppendorf tubes on ice and neutralized with 0.1N NaOH to pH 7.4.

Through the methods described above, a native rat cardiac ECM gel has been formed. Successful gelation of 2.5-8 mg/mL gels occurs within 15 min, as confirmed by the increased viscous nature of the material. Increased stiffness is observed with higher ECM density gels. The pH-adjusted solution is diluted to concentration with 1×PBS, plated on a 96 well plate at 50 μL per well, and then transferred to an incubator at 37° C. and 5% CO2. After the gel has formed, 100 μL of isolated 2 d neonatal cardiomyocyte cells are pipetted on top of the gel at 60,000 cells per well. After a few days, cells are examined for adherence to the gels.

Plating cardiomyocytes on the cardiac ECM gels at 1×10⁴ shows successful adhesion and survival of cells to the ECM. The cells are cultured on the ECM for up to four days.

One hundred mL of cardiac ECM solution (7 mg/mL) is injected through a 30 G needle into the LV free wall of an anesthetized rat. In summary, the study shows that native heart extracellular matrix can be isolated, solubilized, and self-assembled into a gel when brought to physiological pH and temperature.

### Example 2

Here, cell coating use has been investigated for native heart extracellular matrix of adult ventricles that have been decellularized and solubilized. The advantages being that native heart ECM may have more components than traditional cell coatings, and be more readily available for use than pretreatment with other cell types.

Hearts are removed from Sprague-Dawley rats, and decellularized. The decellularized hearts are lyophilized, rehydrated, and pulverized after freezing in liquid nitrogen. The ECM is then digested in pepsin in 0.1M HCl. After 48 hours of digestion, 0.01 M acetic acid is added to dilute to the final concentration of 1 mg/ml.

Pepsin digestion of the native heart ECM is run in vertical gel electrophoresis in reducing conditions using dithiothreirol (DTT) and compared against laminin (BD Biosciences), and calf skin collagen (Sigma). Gels are stained with Imperial Protein Stain (Pierce). Native heart ECM can demonstrate a more complex mixture of ECM components when compared to collagen and laminin.

Cardiac myocytes are harvested from the ventricles of 1 to 2 day old Sprague-Dawley rats using an isolation kit (Cellutron, Highland Park, N.J.). The initial supernatant is discarded, but the subsequent 20 min digestions are strained and suspended in DMEM supplemented with 17% M199, 10% horse serum, 5% fetal bovine serum, and 1% penicillin/streptomycin. After isolation, the supernatant is pre-plated onto tissue culture polystyrene dishes to increase purity of cardiomyocytes through selective adhesion of fibroblasts.

Either 1 mg/ml native cardiac ECM or Collagen I (Sigma, St. Louis, Mo.) is adsorbed onto tissue culture 48-well plates for 1 hour at 37° C. Isolated neonatal cardiomyocytes are plated at a density of 200,000/cm2 and media is changed to low serum maintenance media after 24 hours (DMEM, 18.5% M199, 5% HS, 1% FBS and 1% penicillin/streptomycin). Cell cultures are maintained at 37° C. and 5% carbon dioxide, monitored daily, and fresh media is added every 2-3 days. Cultures are fixed at day 2, day 4, and day 7 and stained for alpha actinin, connexin43, pan-cadherin, actin and nuclei. Cardiomyocytes begin to spontaneously beat in culture at Day 2. Cells cultured on collagen begin detaching from the plate at Day 8. One set of cells cultured on native heart ECM continue beating until Day 45. All cells cultured on collagen stop beating at Day 14.

The native cardiac ECM is shown by this study to contain more complex components when compared to other standard cell culture coatings. Neonatal rat cardiomyocytes attach to native heart ECM as a coating for cell culture, spontaneously began beating. Cardiomyocytes cultured on native cardiac ECM demonstrated increase actinin, connexin43, and pan-cadherin staining over time. Also, the neonatal cardiomyocytes have increased survivability and attachment on the native heart ECM when compared to collagen.

### Example 3

In vitro and in vivo chemoattractive properties of the cardiac decellularized ECM solution are tested. In vitro chemoattractive properties are tested using a commercially available migration assay kit. Human coronary artery endothelial cells and rat aortic smooth muscle cells are serum starved and migration is evaluated towards the matrix, collagen, pepsin and fetal bovine serum. Rat aortic smooth muscle cells show significant migration towards the matrix, while human coronary artery endothelial cells show a trend of migration towards the matrix.

In vivo, cardiac decellularized ECM solution is injected. The arteriole formation is quantified within the injected region to assess neovascular formation. Arteriole density is significantly greater at 11 days post injection, as compared to 4 hours post injection. Thus, the biochemical cues of the matrix have chemoattractive properties that can promote cell infiltration in vivo.

### Example 4

Myocardial infarction is induced in rats using a 25 min ischemia-reperfusion model, via occlusion of the left anterior descending artery. At one week post-MI baseline function is calculated from MRI images. Porcine myocardial ECM is decellularized in small pieces, in 1% SDS for several days, followed by a DI rinse overnight, lyophilization and milling to create a powder. Digestion is performed in 0.1 M HCl with pepsin to create a solubilized form of the material.

Solubilized ECM is brought to pH 7.4 using 1 M NaOH and diluted with PBS to be 6 mg/mL prior to injection. After MI surgery, animals are randomized into two groups and ECM or saline is injected into the LV free wall of female Sprague Dawley rats through a 30 G needle, two weeks after infarction surgery.

4 weeks after injection surgery (6 weeks post-MI), cardiac function is again assessed using MRI.

Animals injected with ECM show preserved function (as evaluated based on ejection fraction) at 6 weeks, while saline injected animals do not maintain cardiac function. End diastolic and end systolic volume are also preserved in ECM injected animals.

### Example 5

Currently, stem cells and other cell types are in clinical trials for treatment of myocardial infarction by delivery through a 27 G catheter into the myocardial wall. Porcine ventricular tissue is decellularized using SDS detergents, and processed to form a solubilized form of the matrix, and neutralized to physiologic pH and diluted to 6 mg/mL for injection.

Two Yorkshire pigs receive a coil-induced myocardial infarction and are injected with myocardial matrix alone or with cells at two months post infarction.

Derived from fetal cardiac explants are pre-labeled with a fluorescent dye, 1,1′-Dioctadecyl 3,3,3′,3′ tetramethylindocarbocyanine iodide (DiI), which is a cytoplasmic stain, for histological identification. A pro-survival cocktail, shown to enhance hESC survival in a rodent model, is used.

Matrix alone or with cells is injected at a clinically relevant rate of 0.2 mL per 30 seconds through a catheter, as guided by NOGA mapping. 5 injections of 0.1 mL each are made of matrix alone or with cells into border zone regions of the infarct.

Matrix alone and matrix with cells are able to be successfully injected into the porcine heart, minimally invasively, without clogging the narrow catheter.

### Example 6

Porcine ventricular myocardium is decellularized, and cell removal is confirmed by hematoxylin and eosin (H&E) staining of fresh frozen decellularized tissue sections. Following this decellularization procedure, the ECM is lyophilized and then milled into a fine particulate. The myocardial ECM powder is characterized using Liquid Chromatography Mass Spectrometry (LC-MS/MS), which allows for the identification of proteins and proteoglycans. LC-MS/MS revealed a variety of ECM proteins, indicating retained protein content after decellularization. The ECM proteins, glycoproteins, and proteoglycans identified include: collagen types I, III, IV, V, and VI, elastin, fibrinogen, lumican, perlecan, fibulin, and laminin. The identification of these components within the decellularized myocardial ECM indicates a retained complex combination of proteins and proteoglycans.

To generate the injectable form of the composition, decellularized matrix powder is solubilized, or partially digested through enzymatic digestion. The matrix is allowed to digest for 48 hr under constant stirring. It is determined that the glycosaminoglycan (GAG) content of the solubilized product is 23.2±4.63 μg per mg of matrix. The collagen content of the solubilized product is higher than 1000 ug/ml.

### Example 7

The liquid composition is brought up to physiologic pH through the addition of NaOH and 10×PBS, and diluted to its final concentration with 1×PBS. At this point, the product can be used immediately, or can be lyophilized, stored frozen, and rehydrated with sterile water prior to use.

The composition self-assembles into a hydrogel upon transendocardial injection in vivo into 25 injection sites (0.2 mL each site) throughout the septal wall and LV free wall. Detection of the matrix within the LV free wall and septal wall confirms successful delivery into the myocardium, as well as gelation of the matrix in vivo. No material is observed in satellite organs.

### Example 8

A composition prepared according to Examples 6 and 7 (referred to in this example as “Composition”) is used in the present example. Composition (n=6) or saline (n=6) is injected into the LV free wall of female Sprague Dawley rats two weeks after infarction. Magnetic resonance imaging (MRI) is used to assess cardiac function and LV geometry one week post-MI, as a pre-treatment baseline, and at six weeks post-MI. Both the LV volume and ejection fraction at four weeks post-injection remain statistically equivalent to baseline measurements in composition injected animals, whereas both worsen in the saline control animals as demonstrated in Table 1. The LV volume and ejection fraction at four weeks post-injection remain statistically equivalent to baseline measurements in injected animals, whereas both worsen in the saline control animals (*P<0.05 compared to baseline; § P=0.054). There are also trends in improvement in the percent changed in EF and volumes.

TABLE 1
MRI data

| | |
|---|---|
| 1 week post-MI | 4 weeks |
| (1 week pre-injection) | post-injection |

Ejection Fraction

| | | |
|---|---|---|
| Saline | 58 ± 6% | 55 ± 11%* |
| Composition | 62 ± 5% | 62 ± 9% |

End Diastolic Volume (mm³)

| | | |
|---|---|---|
| Saline | 325 ± 49 | 451 ± 90* |
| Composition | 331 ± 66 | 414 ± 45 |

End Systolic Volume (mm³)

| | | |
|---|---|---|
| Saline | 137 ± 32 | 205 ± 61* |
| Composition | 126 ± 34 | 157 ± 37 |

*p < 0.05: indicates significant difference compared to baseline measurements. Paired comparisons were performed to allow for each animal to be its own internal control because of the high variability between animals in this model.

### Example 9

Porcine ventricular myocardium is decellularized and cell removal is confirmed by hematoxylin and eosin (H&E) staining of fresh frozen decellularized tissue sections, staining with Hoechst 33342, and through a DNEasy kit. Following this decellularization procedure, the ECM is lyophilized and then milled into a fine particulate. The myocardial ECM powder is characterized using Liquid Chromatography Mass Spectrometry (LC-MS/MS), which allows for the identification of proteins and proteoglycans. LC-MS/MS reveals a variety of ECM proteins, indicating retained protein content after decellularization. The ECM proteins, glycoproteins, and proteoglycans identified include, without limitation: collagen types I, III, IV, V, and VI, elastin, fibrinogen, lumican, perlecan, fibulin, and laminin. The identification of these components indicates a retained complex combination of proteins and proteoglycans.

To generate the injectable form, decellularized matrix powder is processed into a liquid through enzymatic digestion. The matrix is allowed to digest for 48 hr under constant stirring, yielding liquid. Complete digestion is confirmed by lack of visible particles in solution, as well as the presence of low molecular weight species with gel electrophoresis. In some instances, a composition herein lacks nuclei/DNA, has molecular weight bands below 20 kDa, has a GAG content between 15-25 μg/mg of matrix, and lack of visible particles after 48 hr of digestion. Liquid is brought up to physiologic pH through the addition of NaOH and 10×PBS, and diluted to its final concentration (6 mg/mL, which has already been optimized for appropriate gelation characteristics) with 1×PBS. At this point, the product can be used immediately, or can be lyophilized, stored frozen, and rehydrated with sterile water prior to use. To induce gelation in vitro, the solution is brought up to 37° C., which forms a porous and fibrous scaffold similar in scale and structure as native ECM. Or the material can also be injected in vivo where it self-assembles into a hydrogel.

### Example 10

Here, the investigation and use of a decellularized pericardial tissue is described as pertaining to its potential as an autologous therapy to improve cell retention and survival in the LV wall by promoting neovascularization in vivo.

Both porcine and human pericardia have been decellularized. Juvenile male farm pigs are euthanized and their pericardia are decellularized. Specifically, pericardia are rinsed briefly in DI water, stirred in 1% SDS for 24 hours, and then stirred in DI water for approximately 5 hours. Human pericardial tissue samples are collected from patients undergoing cardiothoracic surgeries. The samples are decellularized by a brief DI water rinse, 3 days in 1% SDS, followed by an overnight DI rinse. Complete decellularization for both cases is verified with histology.

Decellularized pericardia or pericardial ECM are then frozen, lyophilized, and milled to form a fine, dry powder. The ECM powder is digested with pepsin dissolved in HCL and neutralized. Gel electrophoresis (SDS-PAGE) indicates greater complexity than in pepsin-digested collagen, showing a wide range of smaller bands in the pericardium samples. This complexity is confirmed by analyzing the samples with mass spectroscopy to identify protein fragments. Fragments of ECM proteins identified included collagen, elastin, fibrin, and a variety of proteoglycans.

Neutralized pericardial ECM solution transforms into a gel after 2-3 hours when 150 ul of the neutralized pericardial ECM solution is loaded into a 96 well plate and allowed to stand in an incubator.

In vivo gelation is observed by injecting 60 ul of the neutralized ECM solution into the LV wall of male Sprague Dawley rats. Histological staining of hearts sectioned from animals sacrificed 45 minutes after injection show an area of felled ECM visible in the LV wall.

Animals are maintained for two weeks, after which they are sacrificed and their hearts are harvested for analysis. The ECM gel injection is still visible at this time point and has been infiltrated by cells Immunohistochemistry is performed on tissue slices in order to identify the smooth muscle cells and endothelial cells, indicative of blood vessels. The presence of a large number of vessels within the ECM injection area indicates that the ECM biomaterial promotes neovascularization.

### Example 11

Here, the use of a gel made from native decellularized skeletal muscle ECM is described. Porcine skeletal muscle is decellularized. The tissue is sliced to be about 2 mm thick and is rinsed with DI water, then stirred in 1% SDS in PBS until decellularization is complete. Decellularized tissue is then rinsed in DI water to ensure removal of detergents. Pieces of decellularized tissue are sectioned and stained using hematoxylin and eosin to ensure removal of cells. Decellularized tissue is then lyophilized and milled to form a fine powder.

The skeletal muscle ECM is digested in pepsin in low acidic conditions, and neutralized to physiological or near physiological pH through the addition of sodium hydroxide and 10×PBS. Neutralized skeletal muscle ECM solution is then diluted with PBS to the desired concentration of 6 mg/ml and allowed to gel in 96 well plates at 37 degree C. Successful gelation is confirmed by visual inspection of the material.

Solubilized native skeletal muscle ECM at a concentration of 6 mg/ml is successfully injected through a 25 G needle into rat leg femoral muscle creating a gelled scaffold. Gelation occurred within 10-15 minutes after being injected in vivo. Muscle and ECM is excised, sectioned and stained using hematoxylin and eosin to confirm successful gelation of skeletal muscle ECM in the muscle.

Skeletal muscle ECM can also be used to deliver cells, such as skeletal myoblast or other muscle relevant cell types in the ECM.

### Example 12

A composition prepared according to Examples 6 and 7 (referred to in this example as “Composition”) is used in the present example. Composition (n=6) or saline (n=6) is injected into the LV free wall of female Sprague Dawley rats two weeks after infarction. Warfarin, in a dosage that is equivalent to 5 mg clinical dosage in human, is administered orally to the rats once a day. Overall there are four groups of rats, n=6 each: composition plus warfarin treatment, composition alone, saline plus warfarin treatment, and saline alone. Magnetic resonance imaging (MRI) is used to assess cardiac function and LV geometry one week post-MI, as a pre-treatment baseline, and at six weeks post-MI.

Both the LV volume and ejection fraction at four weeks post-injection remain statistically equivalent to baseline measurements in composition injected animals with or without warfarin treatment, whereas both worsen in the saline control animals with or without warfarin treatment. There are also trends in improvement in the percent changed in EF and volumes. Generally, there are improvements in LV volume and ejection fraction for composition-injected animals compared to warfarin-only treated animals.

### Example 13

Porcine ventricular myocardium is decellularized, and cell removal is confirmed by hematoxylin and eosin (H&E) staining of fresh frozen decellularized tissue sections. Following this decellularization procedure, the ECM is lyophilized and then milled into a fine particulate. The myocardial ECM powder is characterized using Liquid Chromatography Mass Spectrometry (LC-MS/MS), which allows for the identification of proteins and proteoglycans. LC-MS/MS revealed a variety of ECM proteins, indicating retained protein content after decellularization. To generate the injectable form of the composition, decellularized matrix powder is solubilized through enzymatic digestion. The liquid composition is brought up to physiologic pH through the addition of NaOH and 10×PBS, and diluted to its final concentration with 1×PBS.

ECM composition or saline is injected into the LV peri-infarct region of the wall of the hearts of pigs two weeks after infarction. Separately, coronary stents are implanted into the pigs. Overall, there are four groups (n=6 each): ECM+stent, ECM alone, stent alone and saline. Magnetic resonance imaging (MRI) is used to assess cardiac function and LV geometry one week post-MI, as a pre-treatment baseline, and at six weeks post-MI.

### Example 14

This was a pre-clinical study to investigate biodistribution. A composition prepared according to Examples 6 and 7 (referred to in this example as “Composition”) is used in the present example.

Biotin-labeled composition was tested. Two female Yucatan mini pigs were used for the studies. Twenty five or fifteen 0.25-ml injections were performed in the infarct and border zone. 1 to 2 h following injection, pigs were euthanized, sacrificed and the heart was removed. Heart slices were fresh frozen for histological analysis, to locate the composition. In addition, tissue of the following organs was collected to assess distribution to satellite organs: right and left lungs, liver, spleen, right and left kidneys, and right and left brain. Samples were frozen and H&E stained for histological analysis. Adjacent sections were stained for visualization of biotin-labeled composition.

The composition was observed to gel in vivo and there were no signs of pericardial effusion. Histological analysis of other organs confirmed the gelling remained localized to the cardiac tissue into which the composition was injected.

### Example 15

This was a pre-clinical study to investigate efficacy. Two weeks post-infarction, injections of the composition and saline (control) were delivered using a catheter. The composition or saline was injected using a electromapping to guide injections throughout the infarct and border zone. The composition was applied by 14-15 injections of 0.25 mL for a total of 3.5-3.75 mL in each animal. Animals were maintained for three months. Clinical laboratory assays and Holter monitoring were used to evaluate animal health and determine potential effects on cardiac rhythm. Echocardiography was performed at baseline, prior to injection, and again prior to euthanasia.

ECGs were taken at the time of each procedure and Holter monitoring was performed for 24 h at each of the following time points: prior to MI, prior to injection, one week p. i., one month p. i., and approximately one week prior to euthanasia.

Echocardiography data showed improvements in cardiac function parameters in the composition-injected animals and a worsening of function in the saline control animals. Parameters measured include ejection fraction (EF), LV end diastolic (EDV) and end systolic volumes (ESV). After three months post-infarction, EF of the composition group was significantly greater, and EDV and ESV were significantly smaller than those of the saline control animals.

### Example 16

Safety pharmacology of VentriGel was examined in rats and pigs with myocardial infarction and VentriGel or control treatment. In rats, the inducibility of arrhythmia via external electrical stimulation was compared, while in pigs ECGs were recorded during the efficacy study to evaluate cardiovascular safety. No cardiovascular risk associated with VentriGel-treatment was identified in these studies. Also further ECG recordings in other pig studies did not reveal any proarrhythmogenic potential of VentriGel.

### Example 17

This was a study to investigate myocardial infarction.

Cardiovascular disease continues to be the leading cause of death in the United States, as well as the rest of the western world, with an estimated 785,000 new myocardial infarctions (MIs) each year. Post-MI pathological changes are often progressive, consisting of an initial inflammatory phase, followed by the up-regulation of matrix metalloproteinases that degrade ECM, leading to infarct expansion and wall thinning, and eventual collagen scar deposition to resist deformation and rupture. The resultant negative left ventricular (LV) remodeling is thought to independently contribute to progressive deterioration of cardiac function leading to heart failure post-MI. When end-stage failure occurs, heart transplantation or implantation of an LV assist device are the only available treatments. Therefore, the development of new therapies is necessary.

One of the first alternatives to heart transplantation was a technique termed cellular cardiomyoplasty. This technique consists of injecting cells, suspended in saline or cell culture medium, into the recipient's myocardium (Dib, Diethrich et al. 2002; Fuchs, Satler et al. 2003; Perin, Dohmann et al. 2003; Smits, van Geuns et al. 2003; Dib, Michler et al. 2005; Dib, Campbell et al. 2006; Fuchs, Kornowski et al. 2006). This is an attractive approach because it allows for minimally invasive intramyocardial delivery through a catheter. Although many clinical trials using cellular cardiomyoplasty have shown some promise (Fuchs, Satler et al. 2003; Perin, Dohmann et al. 2003; Smits, van Geuns et al. 2003; Dib, Michler et al. 2005; Fuchs, Kornowski et al. 2006), cell retention, engraftment, and survival have been difficult to achieve, due in part to the lack of an appropriate extracellular microenvironment (Davis, Hsieh et al. 2005).

The compositions herein are cardiac-specific injectable materials, offering a replacement scaffold that mimics the native cardiac extracellular environment. It can eliminate many of the complications associated with cell therapies. The ventricular ECM is processed into an injectable liquid that self-assembles upon injection in vivo to form a nanofibrous and porous scaffold.

The composition is injectable into patients with an acute ST-elevation myocardial infarction (STEMI) within the prior 7 to 21 days. In some cases, the patients had a reperfusion intervention (e.g. percutaneous coronary intervention devices). Patients treated by the composition have a left ventricular ejection fraction (EF) of greater than or equal to 35% but less than or equal to 45% as measured by echocardiography during the screening period (3-5 days post index event). Patients with EF of less than 35% or greater than 45% are also evaluated.

When treating a patient with an MI, an electromechanical cardiac mapping system can be used to map the damaged left ventricle and then an injection (e.g. using a transendocardial catheter injection system or direct injection) can be made into the left ventricle, either directly into the infarct, the border zone of the infarct, or the healthy tissue surrounding the infarct.

Within months after the injection, the myocardial infarction size shrinks as compared to pre-injection. The left ventricular dimensions and end diastolic and end systolic volumes are measured Changes in the ventricular dimensions and EDV and ESV are significantly less than no intervention to the left ventricle or compared to minimal intervention (e.g. saline injections). In addition, the wall motion scores of the left ventricle is more similar to normal as compared to no intervention to the left ventricle or compared to minimal intervention (e.g. saline injections).

In some instances, patients are 30 to 75 years old. In other instances, patients are younger than 30 years old or older than 75 years. In some instances, patients have TIMI II flow scores before receiving an intervention.

In some instances, patients treated with the compositions herein do not enter heart failure after MI within 5 years. In other instances, MI patients treated with the composition herein never enter heart failure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a subject with an acute ST-elevation myocardial infarction (STEMI) comprising injecting a biomaterial to the heart of the subject, wherein the total volume of the biomaterial injected is 1-10 ml.

2. The method of claim 1, wherein the biomaterial is injected into the infarct of the subject.

3. The method of claim 1, wherein the injecting of the biomaterial is provided through a cardiac catheter with a femoral artery access.

4. The method of claim 1, wherein the subject has a left ventricular ejection fraction of 25%-55%.

5. The method of claim 1, wherein the biomaterial comprises extracellular matrix (ECM) derived from a cardiac tissue.

6. The method of claim 1, further comprising conducting a diagnostic imaging procedure to the subject prior to the injection.

7. The method of claim 6, wherein the diagnostic imaging procedure is conducted less than 2 weeks after the myocardial infarction.

8. The method of claim 1, wherein the subject is separately delivered a therapeutic cardiac device.

9. The method of claim 8, wherein the therapeutic cardiac device is a percutaneous coronary intervention device.

10. The method of claim 1, wherein the subject is further administered one or more therapeutic agents that are configured to reduce a heart load.

11. The method of claim 10, wherein the one or more therapeutic agents are selected from the group consisting of a blood pressure medication, an antiarrhythmic medication, a cholesterol-lowering drug, a blood thinner, an anticoagulant, a medication that controls heart rate, and a vasodilator.

12. The method of claim 11, wherein the biomaterial and the one or more therapeutic agents are delivered separately.

13. The method of claim 1, wherein the subject shows improvement in ejection fraction after the injection of the biomaterial.

14. The method of claim 1, wherein the biomaterial increases cell influx in the infarct.

15. The method of claim 1, wherein the left ventricular geometry of the subject is preserved.

16. The method of claim 1, wherein after the injection of the biomaterial, the subject does not enter heart failure within 5 years.

17. The method of claim 8, wherein the subject shows improvement in cardiac function after the injection of the biomaterial, and wherein the improvement of the cardiovascular condition is greater than the improvement from treating the subject with the biomaterial alone or the therapeutic cardiac device alone.

18. The method of claim 10, wherein the subject shows improvement in cardiac function after the injection of the biomaterial, and wherein the improvement is greater than the improvement from treating the subject with the biomaterial alone or with the one or more therapeutic agents alone.

19. The method of claim 8, wherein the biomaterial and the therapeutic cardiac device act upon the cardiovascular condition synergistically.

20. The method of claim 10, wherein the biomaterial and the one or more therapeutic agents act upon the cardiovascular condition synergistically.

21. A method of delivering extracellular matrix (ECM) to a subject comprising:
a. delivering a first composition in a liquid form to the subject, wherein the first composition comprises the ECM or components derived from the ECM; and
b. delivering a second composition in a liquid to the subject such that a portion of the second composition contacts a portion of the first composition within the subject;
wherein the ECM forms a gel after the contacting of the first composition with the second composition.
